(19) United States
(12) Patent Application Publication
(10) Pub. No.: US 2006/0094782 A9
(43) Pub. Date: May 4, 2006

(54) Compositions and dosage forms for enhanced absorption

(76) Inventors: Patrick S.L. Wong, Burlingame, CA (US); Dong Yan, Cupertino, CA (US); George V. Guittard, Cupertino, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/978,139

(22) Filed: Oct. 29, 2004

Related U.S. Application Data
(60) Provisional application No. 60/519,509, filed on Nov. 12, 2003.
(60) Provisional application No. 60/516,259, filed on Oct. 31, 2003.

(51) Int. Cl. A61K 31/185; B65D 88/76

(52) U.S. Cl. 514/553; 514/560; 220/484

(57) ABSTRACT

Disclosed is controlled delivery of pharmaceutical agents and methods, dosage forms and devices therefore. In particular, formulation, dosage forms, methods and devices for enhanced absorption and controlled delivery drug compounds are disclosed.

### CROSS-REFERENCE TO RELATED APPLICATIONS

This application claims the benefit of U.S. provisional patent application No. 60/519,509, filed Nov. 12, 2003, and of U.S. Provisional Application No. 60/516,259, filed Oct. 31, 2003, both applications are incorporated herein by reference in their entirety.

### FIELD OF THE INVENTION

This invention relates to the controlled delivery of pharmaceutical agents and methods, dosage forms and devices therefore. In particular, the invention is directed to formulations, dosage forms, methods, and devices for enhanced absorption and controlled delivery of drug compounds.

### BACKGROUND OF THE INVENTION

In conventional pharmaceutical development the choice of dosage forms such as a base or salt is based on obtaining, on the one hand, the most stable dosage form, and on the other, providing maximum absorption in the upper gastrointestinal (G.I.) tract. As most drug dosage forms are designed for immediate release of the drug dosage, the dosage form is made to be well dissolved in the upper G.I. tract and usually highly dissociated, i.e., highly charged, in the G.I. environment of the small and large intestines (pH=approximately 5-7).

Pharmaceutical development also typically targets drug forms for absorption in the upper G.I. tract, rather than the lower G.I. tract, because of the far greater surface area for absorption of drugs in the upper G.I. tract. The lower G.I. tract lacks microvilli which are present in the upper G.I. tract. The presence of microvilli greatly increases the surface area for drug absorption, and the upper G.I. tract has 480 times the surface area than does the lower G.I. tract. Differences in the cellular characteristics of the upper and lower G.I. tracts also contribute to the poor absorption of molecules in the lower G.I tract.

FIG. 1 illustrates two common routes for transport of compounds across the epithelium of the G.I. tract. Individual epithelial cells, represented by 10a, 10b, 10c, form a cellular barrier along the small and large intestine. Individual cells are separated by water channels or tight junctions, such as junctions 12a, 12b. Transport across the epithelium occurs via either or both a transcellular pathway and a paracellular pathway. The transcellular pathway for transport, indicated in FIG. 1 by arrow 14, involves movement of the compound across the wall and body of the epithelial cell by passive diffusion or by carrier-mediated transport. The paracellular pathway of transport involves movement of molecules through the tight junctions between individual cells, as indicated by arrow 16. Paracellular transport is less specific but has a much greater overall capacity, in part because it takes place throughout the length of the G.I. tract. However, the tight junctions vary along the length of the G.I tract, with an increasing proximal to distal gradient in effective ‘tightness’ of the tight junction. Thus, the duodenum in the upper G.I. tract is more “leaky” than the ileum in the upper G.I. tract which is more “leaky” than the colon, in the lower G.I. tract (Knauf, H. et al., Klin. Wochenschr., 60(19):1191-1200 (1982)).

Since the typical residence time of a drug in the upper G.I. tract is from approximately four to six hours, drugs having poor lower G.I. absorption are absorbed by the body through a period of only four to six hours after oral ingestion. Frequently it is medically desirable that the administered drug be present in the patient's blood stream at a relatively constant concentration throughout the day. To achieve this with traditional drug formulations that exhibit minimal lower G.I. tract absorption, patients would need to ingest the drugs three to four times a day. Practical experience with this inconvenience to patients suggests that this is not an optimum treatment protocol. Accordingly, it is desired that a once daily administration of such drugs, with long-term absorption throughout the day, be achieved.

To provide constant dosing treatments, conventional pharmaceutical development has suggested various controlled release drug systems. Such systems function by releasing their payload of drugs over an extended period of time following administration. However, these conventional forms of controlled release systems are not effective in the case of drugs exhibiting minimal colonic absorption. Since the drugs are only absorbed in the upper G.I. tract and since the residence time of the drug in the upper G.I. tract is only four to six hours, the fact that a proposed controlled release dosage form may release its payload after the residence period of the dosage form in the upper G.I. does not mean that the body will continue to absorb the controlled release drug past the four to six hours of upper G.I. residence. Instead, the drug released by the controlled release dosage form after the dosage form has entered the lower G.I. tract is generally not absorbed and, instead, is expelled from the body.

In response to and in recognition of this, attempts have been made to provide a remedy. These attempts have typically not provided satisfactory results.

Thus, there is a need to develop compounds, methods and products to achieve improved absorption of drugs previously not known to have high absorption throughout the gastrointestinal tract.

### SUMMARY OF THE INVENTION

In an aspect, the invention relates to a substance comprising: a complex that comprises a drug moiety and a transport moiety.

In another aspect, the invention relates to a method of making a composition comprising: providing a drug moiety in an ionic form; providing a transport moiety in an ionic form; combining the drug moiety and the transport moiety, in the presence of a solvent having a dielectric constant less than that of water, to form a complex; and separating the complex from the solvent.

In an aspect, the invention relates to a method of treatment comprising: providing a drug moiety in an ionic form; providing a transport moiety in an ionic form; combining the drug moiety and the transport moiety, in the presence of a solvent having a dielectric constant less than that of water, to form a complex; separating the complex from the solvent; and administering the separated complex to a patient in need thereof.

In another aspect, the invention relates to a method of improving absorption of a drug moiety comprising: providing a complex of the drug moiety and a transport moiety; and administering the complex to a patient in need thereof.

### BRIEF DESCRIPTION OF THE FIGURES

The following figures are not drawn to scale, and are set forth to illustrate various embodiments of the invention.

FIG. 1 is a diagram of epithelial cells of the gastrointestinal tract, illustrating two transport routes of drugs through the epithelium of the G.I. tract.

FIG. 2 shows a diagram of an elementary osmotic pump dosage form.

FIG. 3 shows a diagram of an osmotic dosage form.

FIG. 4 shows a diagram of a tri-layer osmotic dosage form.

FIGS. 5A-5C show diagrams of a controlled release dosage form.

FIG. 6 shows the chemical structure of metformin.

FIG. 7 is a plot of the logarithm of the octanol/water partition coefficient as a function of pH for metformin HCl.

FIG. 8A shows a generalized synthetic reaction scheme for preparation of a metformin-transport moiety complex.

FIG. 8B shows a generalized synthetic reaction scheme for preparation of a metformin-transport moiety complex, where the transport moiety includes a carboxyl group.

FIG. 8C shows a synthetic reaction scheme for preparation of a metformin-fatty acid complex.

FIGS. 9A-9D are HPLC traces of metformin HCl (FIG. 9A), sodium laurate (FIG. 9B), and a physical mixture of metformin HCl, sodium laurate (FIG. 9C), and metformin-laurate complex (FIG. 9D).

FIGS. 10A-10B are plots of conductivity, in microsiemens/centimeter (μS/cm, FIG. 10A) and percent of non-ionized drug (FIG. 10B), as a function of metformin concentration for metformin HCl (circles), metformin complexed with succinate (inverted triangles), caprate (squares), laurate (diamonds), palmitate (triangles), and oleate (octagons).

FIG. 11 shows the metformin plasma concentration, in ng/mL, in rats as a function of time, in hours, for metformin HCl (circles) and a metformin-laurate complex (diamonds) following oral gavage of the compounds to rats.

FIG. 12 shows the metformin plasma concentration, in ng/mL, in rats as a function of time, in hours, for metformin HCl (circles), metformin complexed with succinate (diamonds), palmitate (triangles), oleate (inverted triangles), caprate (squares), and laurate (octagons), using a flush-ligated colonic model.

FIG. 13 shows the percent bioavailability as a function of metformin dose, in mg base/kg, of a physical mixture of metformin HCl and sodium laurate (circles) and of a metformin laurate complex (squares) in rat plasma using a flush-ligated colonic model.

FIG. 14 is a plot of metformin base plasma concentration, in ng/mL, in rats as a function of time, in hours, following intravenous administration of 2 mg/kg metformin hydrochloride (triangles) and following administration of a 10 mg/rat dose of metformin hydrochloride (circles) or metformin laurate complex (diamonds) using a flushed ligated colonic model.

FIG. 15 shows the average release rate of metformin, in mg/hour, as a function of time, in hours for dosage forms according to the invention.

FIG. 16A shows the structure of gabapentin.

FIG. 16B shows the chemical structure of pregabalin.

FIG. 16C shows a synthetic reaction scheme for preparation of a gabapentin-alkyl sulfate complex.

FIG. 16D shows a synthetic reaction scheme for preparation of a pregabalin-alkyl sulfate complex.

FIGS. 17A-17D are FTIR scans of gabapentin (FIG. 17A), sodium lauryl sulfate (FIG. 17B), a physical mixture (loose ionic pair) of gabapentin and sodium lauryl sulfate (FIG. 17C), and gabapentin-lauryl sulfate complex (FIG. 17D).

FIG. 18 shows the gabapentin plasma concentration, in ng/mL, in rats as a function of time, in hours, for gabapentin administered intravenously (triangles) and via intubation into a ligated colon (circles) and for a gabapentin lauryl sulfate complex (diamonds) administered via intubation into a ligated colon.

FIG. 19A shows the gabapentin plasma concentration, in ng/mL, in rats as a function of time, in hours, for gabapentin administered intravenously (triangles) and to the duodenum at dosages of 5 mg (circles), 10 mg (squares) and 20 mg (diamonds).

FIG. 19B shows the gabapentin plasma concentration, in ng/mL, in rats as a function of time, in hours, after administration of gabapentin lauryl sulfate complex intravenously (triangles) and to the duodenum at dosages of 5 mg (circles), 10 mg (squares) and 20 mg (diamonds).

FIG. 19C is a plot of gabapentin bioavailability, in percent, as a function of dose following administration of gabapentin (inverted triangles) or of gabapentin lauryl sulfate complex (circles) to the duodenum of rats.

FIGS. 20A-20C shows a synthetic reaction scheme for preparation of an iron-fatty acid complex.

FIGS. 21A-21D show the structures of exemplary DPP IV inhibitors.

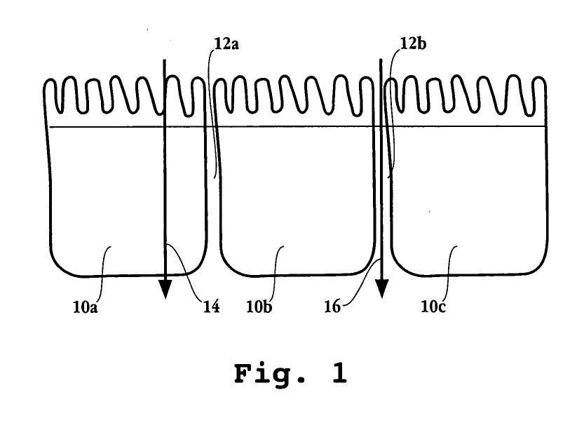

### DETAILED DESCRIPTION

Definitions

The present invention is best understood by reference to the following definitions, the drawings and exemplary disclosure provided herein.

By “composition” is meant one or more of the inventive complexes optionally in combination with additional active pharmaceutical ingredients, and optionally in combination with inactive ingredients, such as pharmaceutically-acceptable carriers, excipients, suspension agents, surfactants, disintegrants, binders, diluents, lubricants, stabilizers, antioxidants, osmotic agents, colorants, plasticizers, and the like.

By “complex” is meant a substance comprising a drug moiety and a transport moiety associated by a tight-ion pair bond. A drug-moiety-transport moiety complex can be distinguished from a loose ion pair of the drug moiety and the transport moiety by a difference in octanol/water partitioning behavior, characterized by the following relationship:
Δ Log D=Log D(complex)−Log D(loose-ion pair)≧0.15 (Equation 1)
wherein:
D, the distribution coefficient (apparent partition coefficient), is the ratio of the equilibrium concentrations of all species of the drug moiety and the transport moiety in octanol to the same species in water (deionized water) at a set pH (typically about pH=5.0 to about pH=7.0) at 25 degrees Celsius. Log D (complex) is determined for a complex of the drug moiety and transport moiety prepared according to the teachings herein. Log D (loose-ion pair) is determined for a physical mixture of the drug moiety and the transport moiety in deionized water. Log D can be determined experimentally or may be predicted for loose-ion pairs using commercially available software packages (e.g., ChemSilico, Inc., Advanced Chemistry Development Inc).

For instance, the octanol/water apparent partition coefficient (D=C_{octanol}/C_{water}) of a putative complex (in deionized water at 25 degree Celsius) can be determined and compared to a 1:1 (mol/mol) physical mixture of the transport moiety and the drug moiety in deionized water at 25 degree Celsius. If the difference between the Log D for the putative complex (D+T−) and the Log D for the 1:1 (mol/mol) physical mixture, D⁺∥T⁻ is determined is greater than or equal to 0.15, the putative complex is confirmed as being a complex according to the invention.

In preferable embodiments, Δ Log D≧0.20, and more preferably Δ Log D≧0.25, more preferably still Δ Log D≧0.35.

The term “DPP IV” as used herein is intended to mean dipeptidyl peptidase IV (EC 3.4.14.5) also known as CD26. A “DPP IV inhibitor” is intended to indicate a molecule that exhibits inhibition of the enzymatic activity of DPP-IV, however the molecule may also have inhibitory activity on other DPP enzymes. A DPP IV inhibitor preserves the action of substrate molecules, including but not limited to GLP-1, GIP, peptide histidine methionine, substance P, neuropeptide Y, and other molecules typically containing alanine or proline residues in the second amino terminal position. In the present context “a DPP IV inhibitor” is also intended to comprise active metabolites and prodrugs thereof. Exemplary DPP IV inhibitors include 1-[[(3-hydroxy-1-adamantyl)amino]acetyl]-2-cyano-(S)-pyrrolidine; 1-{N-(5,6-dichloronicotinoyl)-L-ornithinyl]-3,3-difluoropyrrolidine hydrochloride; and compounds disclosed in WO2004032836; WO2004/024184; WO03/000250, which are incorporated by reference herein; and disclosed in, for example, WO98/19998, DE19616 486 A1, WO00/34241, WO95/15309, WO01/72290, WO01/52825, WO093/10127, WO99/25719, WO99/38501, WO99/46272, WO99/67278 and WO099/67279.

By “dosage form” is meant a pharmaceutical composition in a medium, carrier, vehicle, or device suitable for administration to a patient in need thereof.

By “drug” or “drug moiety” is meant a drug, compound, or agent, or a residue of such a drug, compound, or agent that provides some pharmacological effect when administered to a subject. For use in forming a complex, the drug comprises a(n) acidic, basic, or zwitterionic structural element, or a(n) acidic, basic, or zwitterionic residual structural element. In embodiments according to the invention, drug moieties that comprise acidic structural elements or acidic residual structural elements are complexed with transport moieties that comprise basic structural elements or basic residual structural elements. In embodiments according to the invention, drug moieties that comprise basic structural elements or basic residual structural elements are complexed with transport moieties that comprise acidic structural elements or acidic residual structural elements. In embodiments according to the invention, drug moieties that comprise zwitterionic structural elements or zwitterionic residual structural elements are complexed with transport moieties that comprise either acidic or basic structural elements, or acidic or basic residual structural elements. In an embodiment, the pKa of an acidic structural element or acidic residual structural element is less than about 7.0, preferably less than about 6.0. In an embodiment, the pKa of a basic structural element or basic residual structural element is greater than about 7.0, preferably greater than about 8.0. Zwitterionic structural elements or zwitterionic residual structural elements are analyzed in terms of their individual basic structural element or basic residual structural element or their acidic structural element or acidic residual structural element, depending upon how the complex with the transport moiety is to be formed.

By “fatty acid” is meant any of the group of organic acids of the general formula CH₃(CₙHₓ)COOH where the hydrocarbon chain is either saturated (x=2n, e.g. palmitic acid, CH₃C₁₄H₂₈COOH) or unsaturated (for monounsaturated, x=2n−2, e.g. oleic acid, CH₃C₁₆H₃₀COOH).

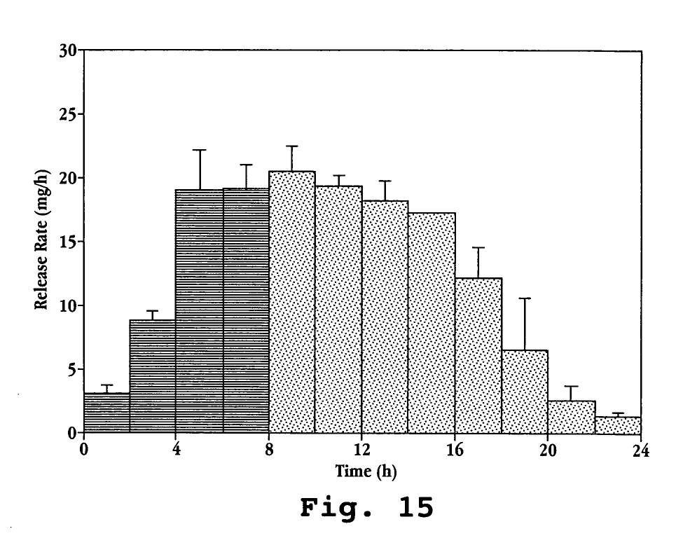

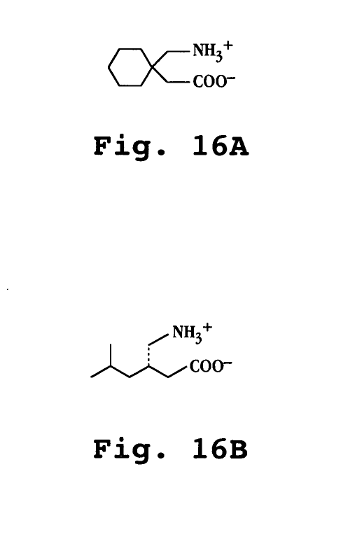

By “gabapentin” refers to 1-(aminomethyl)cyclohexaneacetic acid with a molecular formula of C₉H₁₇NO₂ and a molecular weight of 171.24. It is commercially available under the tradename Neurontin®. Its structure is shown in FIG. 16A.

By “intestine” or “gastrointestinal (G.I.) tract” is meant the portion of the digestive tract that extends from the lower opening of the stomach to the anus, composed of the small intestine (duodenum, jejunum, and ileum) and the large intestine (ascending colon, transverse colon, descending colon, sigmoid colon, and rectum).

By “loose ion-pair” is meant a pair of ions that are, at physiologic pH and in an aqueous environment, are readily interchangeable with other loosely paired or free ions that may be present in the environment of the loose ion pair. Loose ion-pairs can be found experimentally by noting interchange of a member of a loose ion-pair with another ion, at physiologic pH and in an aqueous environment, using isotopic labeling and NMR or mass spectroscopy. Loose ion-pairs also can be found experimentally by noting separation of the ion-pair, at physiologic pH and in an aqueous environment, using reverse phase HPLC. Loose ion-pairs may also be referred to as “physical mixtures,” and are formed by physically mixing the ion-pair together in a medium.

By “lower gastrointestinal tract” or “lower G.I. tract” is meant the large intestine.

By “patient” is meant an animal, preferably a mammal, more preferably a human, in need of therapeutic intervention.

By “pharmaceutical composition” is meant a composition suitable for administration to a patient in need thereof.

By “pregabalin” refers to (S)-(+)-3-(aminomethyl)-5-methylhexanoic acid). Pregabalin is also referred to in the literature as (S)-3-isobutyl GABA or CI-1008. The structure of pregabalin is shown in FIG. 16B.

By “residual structural element” is meant a structural element that is modified by interaction or reaction with another compound, chemical group, ion, atom, or the like. For example, a carboxyl structural element (COOH) interacts with sodium to form a sodium-carboxylate salt, the COO— being a residual structural element.

By “solvent(s)” is meant a substance in which various other substances may be fully or partially dissolved. In the present invention, preferred solvents include aqueous solvents, and solvents having a dielectric constant less than that of water. Preferred solvents having a dielectric constant less than that of water. The dielectric constant is a measure of the polarity of a solvent and dielectric constants for exemplary solvents are shown in Table 1.

TABLE 1
Characteristics of Exemplary Solvents

| Solvent | Boiling Pt., ° C. | Dielectric constant |
|---|---|---|
| Water | 100 | 80 |
| Methanol | 68 | 33 |
| Ethanol | 78 | 24.3 |
| 1-propanol | 97 | 20.1 |
| 1-butanol | 118 | 17.8 |
| acetic acid | 118 | 6.15 |
| Acetone | 56 | 20.7 |
| methyl ethyl ketone | 80 | 18.5 |
| ethyl acetate | 78 | 6.02 |
| Acetonitrile | 81 | 36.6 |
| N,N-dimethylformamide (DMF) | 153 | 38.3 |
| diemthyl sulfoxide (DMSO) | 189 | 47.2 |
| Hexane | 69 | 2.02 |
| Benzene | 80 | 2.28 |
| diethyl ether | 35 | 4.34 |
| tetrahydrofuran (THF) | 66 | 7.52 |
| methylene chloride | 40 | 9.08 |
| carbon tetrachloride | 76 | 2.24 |

The solvents water, methanol, ethanol, 1-propanol, 1-butanol, and acetic acid are polar protic solvents having a hydrogen atom attached to an electronegative atom, typically oxygen. The solvents acetone, ethyl acetate, methyl ethyl ketone, and acetonitrile are dipolar aprotic solvents, and are in one embodiment, preferred for use in forming the inventive complexes. Dipolar aprotic solvents do not contain an OH bond but typically have a large bond dipole by virtue of a multiple bond between carbon and either oxygen or nitrogen. Most dipolar aprotic solvents contain a C—O double bond. Solvents having a dielectric constant less than that of water are particularly useful in the formation of the inventive complexes. The dipolar aprotic solvents noted in Table 1 have a dielectric constant at least two-fold lower than water and a dipole moment close to or greater than water.

By “structural element” is meant a chemical group that (i) is part of a larger molecule, and (ii) possesses distinguishable chemical functionality. For example, an acidic group or a basic group on a compound is a structural element.

By “substance” is meant a chemical entity having specific characteristics.

By “tight-ion pair” is meant a pair of ions that are, at physiologic pH and in an aqueous environment are not readily interchangeable with other loosely paired or free ions that may be present in the environment of the tight-ion pair. A tight-ion pair can be experimentally detected by noting the absence of interchange of a member of a tight ion-pair with another ion, at physiologic pH and in an aqueous environment, using isotopic labeling and NMR or mass spectroscopy. Tight ion pairs also can be found experimentally by noting the lack of separation of the ion-pair, at physiologic pH and in an aqueous environment, using reverse phase HPLC.

By “transport moiety” is meant a compound that is capable of forming, or a residue of that compound that has formed, a complex with a drug moiety, wherein the transport moiety serves to improve transport of the drug across epithelial tissue, compared to that of the uncomplexed drug. The transport moiety comprises a hydrophobic portion and a(n) acidic, basic, or zwitterionic structural element, or a(n) acidic, basic, or zwitterionic residual structural element. In a preferred embodiment, the hydrophobic portion comprises a hydrocarbon chain. In an embodiment, the pKa of a basic structural element or basic residual structural element is greater than about 7.0, preferably greater than about 8.0. Zwitterionic structural elements or zwitterionic residual structural elements are analyzed in terms of their individual basic structural element or basic residual structural element or their acidic structural element or acidic residual structural element, depending upon how the complex with the drug moiety is to be formed.

In a more preferred embodiment, transport moieties comprise pharmaceutically acceptable acids, including but not limited to carboxylic acids, and salts thereof. In embodiments, transport moieties comprise fatty acids or its salts, benzenesulfonic acid or its salts, benzoic acid or its salts, fumaric acid or its salts, or salicylic acid or its salts. In preferred embodiments the fatty acids or their salts, comprise from 6 to 18 carbon atoms (C6-C 18), more preferably 8 to 16 carbon atoms (C8-C16), even more preferably 10 to 14 carbon atoms (C10-C14), and most preferably 12 carbon atoms (C12).

In more preferred embodiments, transport moieties comprise alkyl sulfates (either saturated or unsaturated) and their salts, such as potassium, magnesium, and sodium salts, including particularly sodium octyl sulfate, sodium decyl sulfate, sodium lauryl sulfate, and sodium tetradecyl sulfate. In preferred embodiments the alkyl sulfate or its salt comprise from 6 to 18 carbon atoms (C6-C18), more preferably 8 to 16 carbon atoms (C8-C16), even more preferably 10 to 14 carbon atoms (C10-C14), and most preferably 12 carbon atoms (C12). Also suitable are other anionic surfactants.

In another more preferred embodiment, transport moieties comprise pharmaceutically acceptable primary amines or salts thereof, particularly primary aliphatic amines (both saturated and unsaturated) or salts thereof, diethanolamine, ethylenediamine, procaine, choline, tromethamine, meglumine, magnesium, aluminum, calcium, zinc, alkyltrimethylammonium hydroxides, alkyltrimethylammonium bromides, benzalkonium chloride and benzethonium chloride. Also useful are other pharmaceutically acceptable compounds that comprise secondary or tertiary amines, and their salts, and cationic surfactants.

By “upper gastrointestinal tract” or “upper G.I. tract” is meant that portion of the gastrointestinal tract including the stomach and the small intestine.

Complex Formation and Characterization

It has been surprisingly found that many common drug moieties with poor absorption characteristics, once complexed with certain transport moieties, exhibit significantly enhanced absorption, particularly lower G.I. tract absorption although upper GI tract absorption may also be enhanced. It is further surprising that complexes according to the invention show improved absorption as compared to loose ion-pairs (i.e. a non-complexed form) that comprise the same ions as the inventive complexes.

These unexpected results have been found to apply to many categories of drug moieites, including drug moieties that comprise a basic structural element or a basic residual structural element. Examples of such drug moieties to which the present invention applies include metformin, iron, ranitidine hydrochloride, cetirizine hydrochloride, sumatriptan succinate, oxycodone hydrochloride, tramadol hydrochloride, ciprofaxicin hydrochloride, dipeptidyl peptidase IV (DPP IV) inhibitors, and cimetidine hydrochloride. The unexpected results of the present invention also apply to drug moieties that comprise a zwitterionic structural element or a zwitterionic residual structural element. Examples of such drug moieties to which the present invention applies are gabapentin and levodopa. The unexpected results of the present invention also apply to drug moieties that comprise an acidic structural element or an acidic residual structural element. An example of such a drug moiety to which the present invention applies is rabeprazole sodium.

Examples of preferred embodiments of the present invention are presented below. Presented are preferred embodiments wherein complexes with metformin, iron and gabapentin are formed.

While not wishing to be bound by specific understanding of mechanisms, the inventors reason as follows:

When loose ion-pairs are placed in a polar solvent environment, it is assumed that polar solvent molecules will insert themselves in the space occupied by the ionic bond, thus driving apart the bound ions. A solvation shell, comprising polar solvent molecules electrostatically bonded to a free ion, may be formed around the free ion. This solvation shell then prevents the free ion from forming anything but a loose ion-pairing ionic bond with another free ion. In a situation wherein there are multiple types of counter ions present in the polar solvent, any given loose ion-pairing may be relatively susceptible to counter-ion competition.

This effect is more pronounced as the polarity, expressed as the dielectric constant of the solvent, increases. Based on Coulomb's law, the force between two ions with charges (q1) and (q2) and separated by a distance(r) in a medium of dielectric constant (e) is:

$$F = - \frac{{q}_{1} {q}_{2}}{4 {πɛ}_{0} ɛ {r}^{2}} \qquad ( Equation 2 )$$

where ε₀ is the constant of permittivity of space. The equation shows the importance of dielectric constant (ε) on the stability of a loose ion-pair in solution. In aqueous solution that has a high dielectric constant (ε=80), the electrostatic attraction force is significantly reduced if water molecules attack the ionic bonding and separate the opposite charged ions.

Therefore, high dielectric constant solvent molecules, once present in the vicinity of the ionic bond, will attack the bond and eventually break it. The unbound ions then are free to move around in the solvent. These properties define a loose ion-pair.

Tight ion-pairs are formed differently from loose-ion pairs, and consequently possess different properties from a loose ion-pair. Tight ion-pairs are formed by reducing the number of polar solvent molecules in the bond space between two ions. This allows the ions to move tightly together, and results in a bond that is significantly stronger than a loose ion-pair bond, but is still considered an ionic bond. As disclosed more fully herein, tight ion-pairs are obtained using less polar solvents than water so as to reduce entrapment of polar solvents between the ions.

For additional discussion of loose and tight ion-pairs, see D. Quintanar-Guerrero et al., Applications of the Ion Pair Concept to Hydrophilic Substances with Special Emphasis on Peptides, Pharm. Res. 14(2):119-127 (1997).

The difference between loose and tight ion-pairing also can be observed using chromatographic methods. Using reverse phase chromatography, loose ion-pairs can be readily separated under conditions that will not separate tight ion-pairs.

Bonds according to this invention may also be made stronger by selecting the strength of the cation and anion relative to one another. For instance, in the case where the solvent is water, the cation (base) and anion (acid) can be selected to attract one another more strongly. If a weaker bond is desired, then weaker attraction may be selected.

Portions of biological membranes can be modeled to a first order approximation as lipid bilayers for purposes of understanding molecular transport across such membranes. Transport across the lipid bilayer portions (as opposed to active transporters, etc.) is unfavorable for ions because of unfavorable portioning. Various researchers have proposed that charge neutralization of such ions can enhance cross-membrane transport.

In the “ion-pair” theory, ionic drug moieties are paired with transport moiety counter ions to “bury” the charge and render the resulting ion-pair more liable to move through a lipid bilayer. This approach has generated a fair amount of attention and research, especially with regards to enhancing absorption of orally administered drugs across the intestinal epithelium.

While ion-pairing has generated a lot of attention and research, it has not always generated a lot of success. For instance, ion-pairs of two antiviral compounds were found not to result in increased absorption due to the effects of the ion-pair on trans-cellular transport, but rather to an effect on monolayer integrity. The authors concluded that the formation of ion pairs may not be very efficient as a strategy to enhance transepithelial transport of charged hydrophilic compounds as competition by other ions found in in vivo systems may abolish the beneficial effect of counter-ions. J. Van Gelder et al., “Evaluation of the Potential of Ion Pair Formation to Improve the Oral Absorption of two Potent Antiviral Compounds, AMD3100 and PMPA”, Int. J. of Pharmaceutics 186:127-136 (1999). Other authors have noted that absorption experiments with ion-pairs have not always pointed at clear-cut mechanisms. D. Quintanar-Guerrero et al., Applications of the Ion Pair Concept to Hydrophilic Substances with Special Emphasis on Peptides, Pharm. Res. 14(2):119-127 (1997).

The inventors have unexpectedly discovered that a problem with these ion-pair absorption experiments is that they were performed using loose-ion pairs, rather than tight ion-pairs. Indeed, many ion-pair absorption experiments disclosed in the art do not even expressly differentiate between loose ion-pairs and tight ion-pairs. One of skill has to distinguish that loose ion-pairs are disclosed by actually reviewing the disclosed methods of making the ion-pairs and noting that such disclosed methods of making are directed to loose ion-pairs not tight ion-pairs. Loose ion-pairs are relatively susceptible to counter-ion competition, and to solvent-mediated (e.g. water-mediated) cleavage of the ionic bonds that bind loose ion-pairs. Accordingly, when the drug moiety of the ion-pair arrives at an intestinal epithelial cell membrane wall, it may or may not be associated in a loose ion-pair with a transport moiety. The chances of the ion-pair existing near the membrane wall may depend more on the local concentration of the two individual ions than on the ion bond keeping the ions together. Absent the two moieties being bound when they approached an intestinal epithelial cell membrane wall, the rate of absorption of the non-complexed drug moiety might be unaffected by the non-complexed transport moiety. Therefore, loose ion-pairs might have only a limited impact on absorption compared to administration of the drug moiety alone.

In contrast, the inventive complexes possess bonds that are more stable in the presence of polar solvents such as water. Accordingly, the inventors reasoned that, by forming a complex, the drug moiety and the transport moiety would be more likely to be associated as ion-pairs at the time that the moieties would be near the membrane wall. This association would increase the chances that the charges of the moieties would be buried and render the resulting ion-pair more liable to move through the cell membrane.

In an embodiment, the complex comprises a tight ion-pair bond between the drug moiety and the transport moiety. As discussed herein, tight ion-pair bonds are more stable than loose ion-pair bonds, thus increasing the likelihood that the drug moiety and the transport moiety would be associated as ion-pairs at the time that the moieties would be near the membrane wall. This association would increase the chances that the charges of the moieties would be buried and render the tight ion-pair bound complex more liable to move through the cell membrane.

It should be noted that the inventive complexes may improve absorption relative to the non-complexed drug moiety throughout the G.I. tract, not just the lower G.I. tract, as the complex is intended to improve transcellular transport generally, not just in the lower G.I. tract. For instance, if the drug moiety is a substrate for an active transporter found primarily in the upper G.I., the complex formed from the drug moiety may still be a substrate for that transporter. Accordingly, the total transport may be a sum of the transport flux effected by the transporter plus the improved transcellular transport provided by the present invention. In an embodiment, the inventive complex provides improved absorption in the upper G.I. tract, the lower G.I. tract, and both the upper G.I. tract and the lower G.I. tract.

Complexes according to the invention can be made up of a variety of drug and transport moieties. Generally speaking, the drug moiety is selected first, and then the appropriate transport moiety is selected to form the inventive complex. One of skill could consider a number of factors in selecting transport moieties, including but not limited to the toxicity and tolerability of the transport moiety, the polarity of the structural element or structural element residue of the drug moiety, the strength of the structural element or structural element residue of the drug moiety, the strength of the structural element or structural element residue of the transport moiety, possible therapeutic advantages of the transport moiety. In certain preferred embodiments, the hydrophobic portions of the transport moiety comprises a hydrophobic chain, more preferably an alkyl chain. This alkyl chain may help to promote stability of the complex through sterically protecting the ionic bond from attack by polar solvent molecules.

In preferred embodiments the transport moieties comprise alkyl sulfates or their salts, having from 6 to 18 carbon atoms (C6-C18), more preferably 8 to 16 carbon atoms (C8-C16), even more preferably 10 to 14 carbon atoms (C10-C14), and most preferably 12 carbon atoms (C12). In other preferred embodiments, the transport moieties comprise fatty acids, or their salts, having from 6 to 18 carbon atoms (C6-C18), more preferably 8 to 16 carbon atoms (C8-C16), even more preferably 10 to 14 carbon atoms (C10-C14), and most preferably 12 carbon atoms (C12).

The inventive complexes may be incorporated into a variety of compositions, especially pharmaceutical compositions. In an embodiment, the invention comprises a composition that comprises a complex according to the invention and a pharmaceutically-acceptable carrier. In another embodiment, the invention comprises a pharmaceutical composition that comprises a complex according to the invention and a pharmaceutically-acceptable carrier. Amounts of the complex and other ingredients in the inventive compositions, pharmaceutical compositions, and dosage forms may be determined by one of skill in the art, based on pharmacological and similar requirements. Formulation of such compositions may be performed according to conventional pharmaceutical practices, including milling, mixing, extrusion, compression, coating, and the like.

Complexes according to the invention may be made according to the following general guidelines. Additional strategies may be used such as that strategy exemplified for iron complexes as disclosed in the Examples set forth below.

First, the drug moiety needs to be assessed as to whether it comprises an acidic structural element or an acidic residual structural element that is to form part of the complex (tight ion-pair bond). If so, the next assessment is whether the structural element is acidic or an acid residue. If an acid residue is present, the next step is to determine whether it is a residue of a strong acid or a weak acid. A “weak acid” is a compound having an acid dissociation constant less than about 10⁻⁴. Typically, and as used herein, weak acids are compounds which, when dissolved in water, form mildly acidic solutions, that is, solutions with pH values between about 3-6. Exemplary weak acids include formic acid, acetic acid, propanoic acid, butanoic acid, pentanoic acid, and substituted forms thereof. A “strong acid” typically refers to a compound having an acid dissociation constant of greater than 1. If the residue is characteristic of a strong acid, the drug moiety may be processed using an ion exchange to arrive at the acid form of the drug moiety, which is then isolated using conventional chemical techniques. In an embodiment, the solvent used during the ion exchange comprises a mixture of water and organic solvent. If the residue is that of a weak acid, the drug moiety may be processed using pH titration to reduce ambient pH and arrive at the acid form of the drug moiety, which is then isolated from the aqueous media using conventional chemical techniques.

The acid form of the drug moiety, whether originally present as an acidic structural element or an acidic residual structural element processed as set forth herein to arrive at the acid form of the drug moiety, is then reacted with the transport moiety (which may be present in its basic form) in the presence of a solvent that has a lower dielectric constant than water. Suitable transport moieties comprise those disclosed herein, and preferably comprise cationic surfactants, or amines and salts thereof. The complex is then separated from the solvent.

If the structural element is basic or a basic residue, the next step is to determine whether it is a residue of a strong base or a weak base. Typically, and as used herein, weak bases are compounds which, when dissolved in water, form mildly basic solutions, that is, solutions with pH values between about 8-11. A “strong base” typically refers to a basic compound that is highly dissociated in an aqueous solution. If the residue is that of a strong base, the drug moiety may be processed using an ion exchange to arrive at the base form of the drug moiety, which is then isolated using conventional chemical techniques. In an embodiment, the solvent used during the ion exchange comprises a mixture of water and organic solvent. If the residue is that of a weak base, the drug moiety may be processed using pH titration to raise ambient pH to arrive at the base form of the drug moiety, which is then isolated from the aqueous media using conventional chemical techniques.

The base form of the drug moiety, whether originally present as an basic structural element or an basic residual structural element processed as set forth herein to arrive at the base form of the drug moiety, is then reacted with the transport moiety (which may be present in its acid form) in the presence of a solvent that has a lower dielectric constant than water. Suitable transport moieties comprise those disclosed herein, and preferably comprise fatty acids and their salts, anionic surfactants or other pharmaceutical excipients containing carboxyl groups. The complex is then separated from the solvent.

If the structural element is a zwitterion, or a zwitterionic residue, the next step is to determine whether the acidic or basic group will be the group that forms the complex with the complementary ion on the transport moiety. The group that will not be forming the complex by bonding with the transport moiety may be blocked. A preferred method for blocking the non-bonding structural element or residual structural element is to adjust the environmental pH so that the non-bonding structural element is not ionized. For instance, to block an acidic structural element, the environmental pH is lowered so that the acidic structural element is not ionized, but the basic structural element is. For blocking of basic structural elements, the pH is raised so that the basic structural element is not ionized but the acidic structural element is. Once the desired structural element has been blocked, the drug moiety is isolated, and then reacted with the transport moiety in the presence of a solvent that has a lower dielectric constant than water. The complex is then separated from the solvent.

In an alternative scheme for zwitterionic structural elements or zwitterionic residual structural elements, the transport moiety may be processed using ion exchange to arrive at the acid or base form of the transport moiety, depending upon whether the acidic or basic group will be the group that forms the complex with the complementary ion on the transport moiety. The group that will not be forming the complex by bonding with the transport moiety may be blocked. The acid or base form of the transport moiety may then be reacted with the ionized form of the drug moiety in aqueous media, or a mixture of aqueous media and a solvent having a dielectric constant less than that of water, to form the complex. The complex is then separated from the aqueous media or mixture using conventional chemistry techniques.

In an alternative scheme, use may be made of differing solubility of the counterions of the drug moiety and transport moiety. For instance, if a loose-ion pair made up of the counter-ions is insoluble in water, then it will precipitate out, leaving the drug moiety and the transport moiety in solution. The complex may then be formed, or extracted using a solvent having a dielectric constant less than that of water. An example of this strategy is provided as part of the Iron examples below.

Various solvents can be selected for use in the present invention. Solvents may be selected in part based on physical properties of the drug moiety and/or transport moiety that is to be dissolved therein. Methanol is an exemplary solvent; other solvents are also suitable. For example, fatty acids are soluble in chloroform, benzene, cyclohexane, ethanol (95%), acetic acid, and acetone. The solubility (in g/L) of capric acid, lauric acid, myristic acid, palmitic acid, and stearic acid in these solvents is indicated in Table 2.

TABLE 2
SOLUBILITY (G/L) OF FATTY ACIDS AT 20° C.

| Fatty Acid (no. | | | | | | | | |
|---|---|---|---|---|---|---|---|---|
| carbons) | Chloroform | Benzene | Cyclohexanne | Acetone | Ethanol 95% | Acetic acid | Methanol | Acetonitrile |
| Capric (10) | 3260 | 3980 | 3420 | 4070 | 4400 | 5670 | 5100 | 660 |
| Lauric (12) | 830 | 936 | 680 | 605 | 912 | 818 | 1200 | 76 |
| myristic (14) | 325 | 292 | 215 | 159 | 189 | 102 | 173 | 18 |
| palmitic (16) | 151 | 73 | 65 | 53.8 | 49.3 | 21.4 | 37 | 4 |
| stearic (18) | 60 | 24.6 | 24 | 15.4 | 11.3 | 1.2 | 1 | <1 |

In one embodiment, the solvent used for formation of the complex is a solvent having a dielectric constant less than water, and preferably at least two fold lower than the dielectric constant of water, more preferably at least three-fold lower than that of water.

Solvents, particularly in embodiments wherein a lower dielectric solvent phase and an aqueous phase are present in an admixture, can be selected based, in part, on solvent/molecule interactions. Preferred solvents do not react with either the drug moiety or the transport moiety, and are relatively easy to separate from the complex once the complex is formed. The relative hydrophilicity of the solvent, as compared to the hydrophobicity of the complex, may also be important. If the solvent is too hydrophilic, as compared to the hydrophobicity of the complex, then the complex cannot leave the aqueous phase and enter the solvent phase. The complex, as it is formed, needs to be able to enter the lower dielectric solvent phase (if present), but free ions (with fairly high polarity) should be preferably excluded from the lower dielectric solvent phase (if present).

If the complex is a precipitate, then the complex is isolated by filtration, washing, and drying. If the complex is dissolved, then one or more methods may be utilized: (1) evaporation of the solvent under vacuum conditions, (2) crystallization, or (3) solvent extraction followed by evaporation. The conditions under which these operations are performed may be optimized by one of skill in the art.

Exemplary Dosage Forms and Methods of Use

Complexes according to the invention may be administered to patients in need thereof. In embodiments, the inventive complexes are formulated into dosage forms administerable to patients in need thereof. In preferable embodiments, the complexes are formulated into compositions, more preferably pharmaceutical compositions, of which the dosage forms are comprised.

The complexes described herein provide an enhanced absorption rate in the G.I. tract, and in particular in the lower G.I. tract. Dosage forms and methods of treatment using the complex and its increased colonic absorption will now be described. It will be appreciated that the dosage forms described below are merely exemplary.

A variety of dosage forms are suitable for use with the inventive complexes. A dosage form that permits once daily dosing to achieve a therapeutic efficacy for at least about 12, preferably at least about 15 hours, more preferably for at least 18 hours, and still more preferably for at least about 20 hours, are provided, due the enhanced lower G.I. tract absorption achieved by the complex. The dosage form may be configured and formulated according to any design that delivers a desired dose of the drug moiety. Typically, the dosage form is orally administrable and is sized and shaped as a conventional tablet or capsule. Orally administrable dosage forms may be manufactured according to one of various different approaches. For example, the dosage form may be manufactured as a diffusion system, such as a reservoir device or matrix device, a dissolution system, such as encapsulated dissolution systems (including, for example, “tiny time pills”, and beads) and matrix dissolution systems, and combination diffusion/dissolution systems and ion-exchange resin systems, as described in Remington's Pharmaceutical Sciences, 18th Ed., pp. 1682-1685 (1990).

One important consideration in the practice of this invention is the physical state of the complex to be delivered by the dosage form. In certain embodiments, the inventive complexes may be in a paste or liquid state, in which case solid dosage forms may not be suitable for use in the practice of this invention. In such cases, dosage forms capable of delivering substances in a paste or liquid state should be used. Alternatively, in certain embodiments, a different transport moiety may be used to raise the melting point of the substances, thus making it more likely that the inventive complexes will be present in a solid form.

A specific example of a dosage form suitable for use with the present invention is an osmotic dosage form. Osmotic dosage forms, in general, utilize osmotic pressure to generate a driving force for imbibing fluid into a compartment formed, at least in part, by a semipermeable wall that permits free diffusion of fluid but not drug or osmotic agent(s), if present. An advantage to osmotic systems is that their operation is pH-independent and, thus, continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release, 35:1-21 (1995). Osmotic dosage forms are also described in detail in the following U.S. Patents, each incorporated in their entirety herein: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

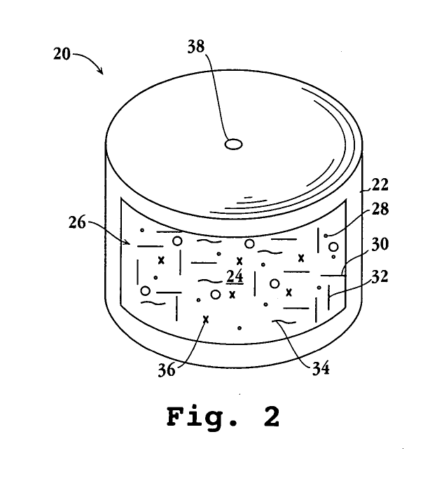

An exemplary dosage form, referred to in the art as an elementary osmotic pump dosage form, is shown in FIG. 2. Dosage form 20, shown in a cutaway view, is also referred to as an elementary osmotic pump, and is comprised of a semi-permeable wall 22 that surrounds and encloses an internal compartment 24. The internal compartment contains a single component layer referred to herein as a drug layer 26, comprising a complex 28 in an admixture with selected excipients. The excipients are adapted to provide an osmotic activity gradient for attracting fluid from an external environment through wall 22 and for forming a deliverable complex formulation upon imbibition of fluid. The excipients may include a suitable suspending agent, also referred to herein as drug carrier 30, a binder 32, a lubricant 34, and an osmotically active agent referred to as an osmagent 36. Exemplary materials for each of these components are provided below.

Semi-permeable wall 22 of the osmotic dosage form is permeable to the passage of an external fluid, such as water and biological fluids, but is substantially impermeable to the passage of components in the internal compartment. Materials useful for forming the wall are essentially nonerodible and are substantially insoluble in biological fluids during the life of the dosage form. Representative polymers for forming the semi-permeable wall include homopolymers and copolymers, such as, cellulose esters, cellulose ethers, and cellulose ester-ethers. Flux-regulating agents can be admixed with the wall-forming material to modulate the fluid permeability of the wall. For example, agents that produce a marked increase in permeability to fluid such as water are often essentially hydrophilic, while those that produce a marked permeability decrease to water are essentially hydrophobic. Exemplary flux regulating agents include polyhydric alcohols, polyalkylene glycols, polyalkylenediols, polyesters of alkylene glycols, and the like.

In operation, the osmotic gradient across wall 22 due to the presence of osmotically-active agents causes gastric fluid to be imbibed through the wall, swelling of the drug layer, and formation of a deliverable complex formulation (e.g., a solution, suspension, slurry or other flowable composition) within the internal compartment. The deliverable complex formulation is released through an exit 38 as fluid continues to enter the internal compartment. Even as drug formulation is released from the dosage form, fluid continues to be drawn into the internal compartment, thereby driving continued release. In this manner, the inventive complex is released in a sustained and continuous manner over an extended time period.

FIG. 3 is a schematic illustration of another exemplary osmotic dosage form. Dosage forms of this type are described in detail in U.S. Pat. Nos. 4,612,008; 5,082,668; and 5,091,190, which are incorporated by reference herein. In brief, dosage form 40, shown in cross-section, has a semi-permeable wall 42 defining an internal compartment 44. Internal compartment 44 contains a bilayered-compressed core having a drug layer 46 and a push layer 48. As will be described below, push layer 48 is a displacement composition that is positioned within the dosage form such that as the push layer expands during use, the materials forming the drug layer are expelled from the dosage form via one or more exit ports, such as exit port 50. The push layer can be positioned in contacting layered arrangement with the drug layer, as illustrated in FIG. 3, or can have one or more intervening layers separating the push layer and drug layer.

Drug layer 46 comprises a complex in an admixture with selected excipients, such as those discussed above with reference to FIG. 2. An exemplary dosage form can have a drug layer comprised of a complex, a poly(ethylene oxide) as a carrier, sodium chloride as an osmagent, hydroxypropylmethylcellulose as a binder, and magnesium stearate as a lubricant.

Push layer 48 comprises osmotically active component(s), such as one or more polymers that imbibes an aqueous or biological fluid and swells, referred to in the art as an osmopolymer. Osmopolymers are swellable, hydrophilic polymers that interact with water and aqueous biological fluids and swell or expand to a high degree, typically exhibiting a 2-50 fold volume increase. The osmopolymer can be non-crosslinked or crosslinked, and in a preferred embodiment the osmopolymer is at least lightly crosslinked to create a polymer network that is too large and entangled to easily exit the dosage form during use. Examples of polymers that may be used as osmopolymers are provided in the references noted above that describe osmotic dosage forms in detail. A typical osmopolymer is a poly(alkylene oxide), such as poly(ethylene oxide), and a poly(alkali carboxymethylcellulose), where the alkali is sodium, potassium, or lithium. Additional excipients such as a binder, a lubricant, an antioxidant, and a colorant may also be included in the push layer. In use, as fluid is imbibed across the semi-permeable wall, the osmopolymer(s) swell and push against the drug layer to cause release of the drug from the dosage form via the exit port(s).

The push layer can also include a component referred to as a binder, which is typically a cellulose or vinyl polymer, such as poly-n-vinylamide, poly-n-vinylacetamide, poly(vinyl pyrrolidone), poly-n-vinylcaprolactone, poly-n-vinyl-5-methyl-2-pyrrolidone, and the like. The push layer can also include a lubricant, such as sodium stearate or magnesium stearate, and an antioxidant to inhibit the oxidation of ingredients. Representative antioxidants include, but are not limited to, ascorbic acid, ascorbyl palmitate, butylated hydroxyanisole, a mixture of 2 and 3 tertiary-butyl-4-hydroxyanisole, and butylated hydroxytoluene.

An osmagent may also be incorporated into the drug layer and/or the push layer of the osmotic dosage form. Presence of the osmagent establishes an osmotic activity gradient across the semi-permeable wall. Exemplary osmagents include salts, such as sodium chloride, potassium chloride, lithium chloride, etc. and sugars, such as raffinose, sucrose, glucose, lactose, and carbohydrates.

With continuing reference to FIGS. 2 or 3, the dosage form can optionally include an overcoat (not shown) for color coding the dosage forms according to dose or for providing an immediate release of the inventive complex or another drug.

In use, water flows across the wall and into the push layer and the drug layer. The push layer imbibes fluid and begins to swell and, consequently, pushes on drug layer 44 causing the material in the layer to be expelled through the exit orifice and into the gastrointestinal tract. Push layer 48 is designed to imbibe fluid and continue swelling, thus continually expelling the inventive complex from the drug layer throughout the period during which the dosage form is in the gastrointestinal tract. In this way, the dosage form provides a continuous supply of complex to the gastrointestinal tract for a period of 12 to 20 hours, or through substantially the entire period of the dosage form's passage through the G.I. tract. Since the complex may be readily absorbed in both the upper and lower G.I. tracts, administration of the dosage form provides delivery of drug moiety into the blood stream over the 12-20 hour period of dosage form transit in the G.I. tract.

In an embodiment, inventive dosage forms comprise inventive complexes and second forms of the drug moiety (such as a loose ion-pair salt) so that the second form of the drug is available for absorption in the upper G.I. tract and the complex is presented for absorption in the lower G.I. tract. This can facilitate optimal absorption in circumstances wherein different characteristics are needed to optimize absorption throughout the G.I. tract.

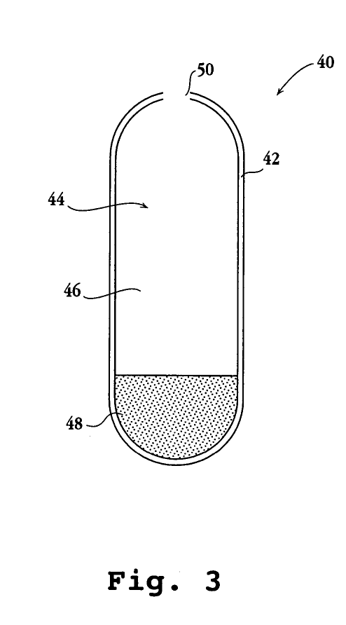

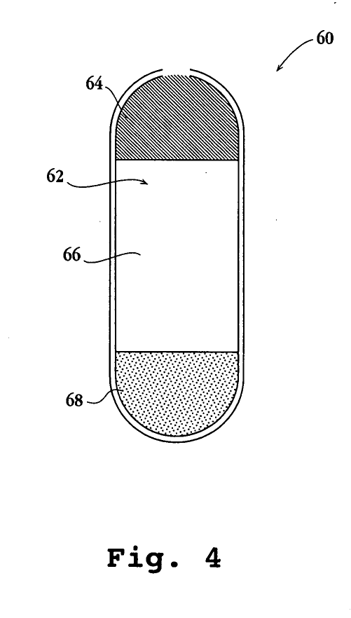

A specific exemplary dosage form comprising the inventive complexes and second forms of the drug moiety (such as a loose ion-pair salt) is shown in FIG. 4. Tri-layered dosage forms of this type are described in detail in U.S. Pat. Nos. 5,545,413; 5,858,407; 6,368,626, and 5,236,689, which are incorporated by reference herein. Osmotic dosage form 60 has a tri-layered core 62 comprised of a first layer 64 of an drug moiety salt, present as a loose ion-pair, a second layer 66 comprising the drug moiety present in the form of an inventive complex, and a third layer 68 referred to as a push layer. A tri-layered dosage form is prepared to have a first layer of 85.0 wt % drug moiety salt present as loose ion-pair salt, 10.0 wt % polyethylene oxide of 100,000 molecular weight, 4.5 wt % polyvinylpyrrolidone having a molecular weight of about 35,000 to 40,000, and 0.5 wt % magnesium stearate. The second layer is comprised 93.0 wt % complex, 5.0 wt % polyethylene oxide 5,000,000 molecular weight, 1.0 wt % polyvinylpyrrolidone having molecular weight of about 35,000 to 40,000, and 1.0 wt % magnesium stearate.

The push layer consists of 63.67 wt % of polyethylene oxide, 30.00 wt % sodium chloride, 1.00 wt % ferric oxide, 5.00 wt % hydroxypropylmethylcellulose, 0.08 wt % butylated hydroxytoluene and 0.25 wt % magnesium stearate. The semi-permeable wall is comprised of 80.0 wt % cellulose acetate having a 39.8 % acetyl content and 20.0 wt % polyoxyethylene-polyoxypropylene copolymer.

Dissolution rates of dosage forms, such as those shown in FIGS. 2-4, can be determined according to procedure set forth in Example 6. In general, release of drug formulation from the dosage form begins after contact with an aqueous environment. In the dosage form illustrated in FIG. 2, release of drug moiety-transport moiety complex, present in the layer adjacent the exit orifice, is released after contact with an aqueous environment and continues for the lifetime of the device. The dosage form illustrated in FIG. 4 provides an initial release of drug moiety salt, present in the drug layer adjacent the exit orifice, with release of drug moiety-transport moiety complex occurring subsequently. It will be appreciated that this dosage form is designed to release drug moiety salt while in transit in the upper G.I. tract, corresponding approximately to the first eight hours of transit. The complex is released as the dosage form travels through the lower G.I. tract, approximately corresponding to times longer than about 8 hours after ingestion. This design takes advantage of the increased lower G.I. tract absorption provided by the complex.

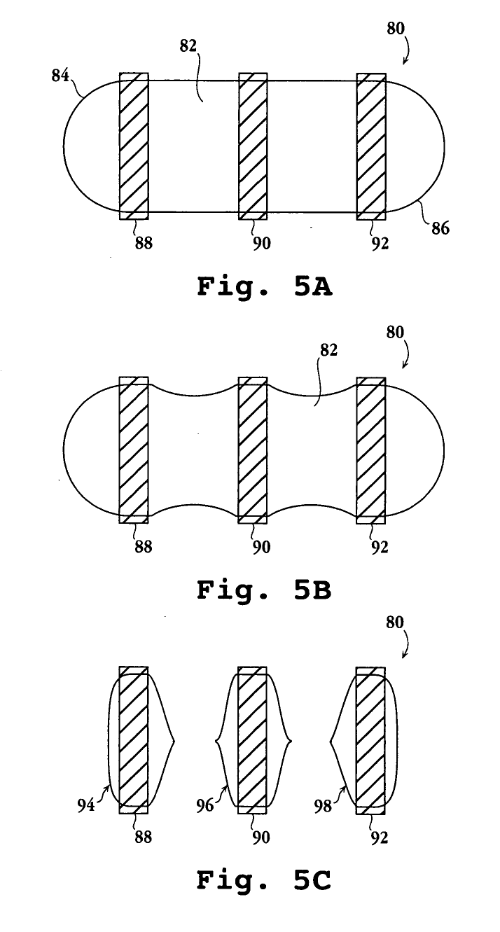

FIGS. 5A-5C illustrate another exemplary dosage form, known in the art and described in U.S. Pat. Nos. 5,534,263; 5,667,804; and 6,020,000, which are specifically incorporated by reference herein. Briefly, a cross-sectional view of a dosage form 80 is shown prior to ingestion into the gastrointestinal tract in FIG. 5A. The dosage form is comprised of a cylindrically shaped matrix 82 comprising an complex. Ends 84, 86 of matrix 82 are preferably rounded and convex in shape in order to ensure ease of ingestion. Bands 88, 90, and 92 concentrically surround the cylindrical matrix and are formed of a material that is relatively insoluble in an aqueous environment. Suitable materials are set forth in the patents noted above and in Example 6 below.

After ingestion of dosage form 80, regions of matrix 82 between bands 88, 90, 92 begin to erode, as illustrated in FIG. 5B. Erosion of the matrix initiates release of the complex into the fluidic environment of the G.I. tract. As the dosage form continues transit through the G.I. tract, the matrix continues to erode, as illustrated in FIG. 5C. Here, erosion of the matrix has progressed to such an extent that the dosage form breaks into three pieces, 94, 96, 98. Erosion will continue until the matrix portions of each of the pieces have completely eroded. Bands 94, 96, 98 will thereafter be expelled from the G.I. tract.

It will be appreciated the dosage forms described in FIGS. 2-5 are merely exemplary of a variety of dosage forms designed for and capable of achieving delivery of the inventive moiety complex to the G.I. tract. Those of skill in the pharmaceutical arts can identify other dosage forms that would be suitable.

The inventive complexes, compositions, and dosage forms are useful in treating a variety of indications. In general, the number of indications treatable using the inventive complexes, compositions, and dosage forms are the same as the number of drug moieties useful in the practice of the invention. In an aspect, the invention provides a method for treating an indication, such as a disease or disorder, in a patient by administering a composition or a dosage form that comprises an inventive complex, the complex characterized by a hybrid bond or a tight ion pair bond between the drug moiety and the transport moiety. In one embodiment, a composition comprising the complex and a pharmaceutically-acceptable vehicle is administered to the patient via oral administration.

The dose administered is generally adjusted in accord with the age, weight, and condition of the patient, taking into consideration the dosage form and the desired result. In general, the dosage forms and compositions comprising the inventive complex may be administered in amounts that provide an amount of the drug moiety within an order of magnitude of the typical immediate release form of the non-complexed drug moiety. Because of the enhanced absorption provided by the complex, the dose of the complex often may be lower than that typically recommended for conventional therapies with the non-complexed drug moiety. Typical doses may comprise drug moiety in an amount ranging from about 0.01 microgram of drug moiety to about 5000 mg of drug moiety, preferably ranging from about 1 microgram of drug moiety to about 2500 mg of drug moiety, more preferably ranging from about 10 micrograms of drug moiety to about 2000 mg of drug moiety, even more preferably ranging from about 100 micrograms of drug moiety to about 1500 mg of drug moiety, and still more preferably ranging from about 500 micrograms of drug moiety to about 1000 mg of drug moiety. Typical doses may comprise the inventive complex in an amount ranging from about 0.01 microgram of the inventive complex to about 5000 mg of the inventive complex, preferably ranging from about 1 microgram of the inventive complex to about 2500 mg of the inventive complex, more preferably ranging from about 10 micrograms of the inventive complex to about 2000 mg of the inventive complex, even more preferably ranging from about 100 micrograms of the inventive complex to about 1500 mg of the inventive complex, and still more preferably ranging from about 500 micrograms of the inventive complex to about 1000 mg of the inventive complex.

From the foregoing, it can be seen how various objects and features of the invention are met. A complex comprising a drug moiety and a transport moiety bound by a tight-ion pair bond or a hybrid bond, may provide an enhanced colonic absorption of the drug moiety, relative to that observed for the non-complexed drug moiety. The complex is prepared from a novel process, where the drug moiety is reacted with a transport moiety, such as a fatty acid, solubilized in a solvent, the solvent being less polar than water, the lower polarity evidenced, for example, by a lower dielectric constant. This reaction results in formation of a complex between the drug moiety and the transport moiety, where the two species are associated by a bond that is not an ionic bond and that is not a covalent bond, but is a tight-ion pair bond.

The invention relates to a substance comprising: a complex that comprises a drug moiety, and a transport moiety. In preferable embodiments, the transport moiety comprises an acidic, a basic, or a zwitterionic structural element; or an acidic, a basic, or a zwitterionic residual structural element paired with an ion in the transport moiety. In preferable embodiments, the transport moiety comprises fatty acids or its salts, benzenesulfonic acid or its salts, benzoic acid or its salts, fumaric acid or its salts, or salicylic acid or its salts. In other preferable embodiments, the fatty acid or its salt comprises a C6-C18 fatty acid or its salt, more preferably the C6-C18 fatty acid or its salt comprises a C12 fatty acid or its salt. In preferable embodiments, the transport moiety comprises an alkyl sulfate or its salt, more preferably the alkyl sulfate or its salt comprises a C6-C18 alkyl sulfate or its salt, more preferably still the C6-C18 alkyl sulfate or its salt is sodium lauryl sulfate. In preferable embodiments, the transport moiety comprises a pharmaceutically acceptable primary, secondary, or tertiary amine, or salts thereof. In more preferable embodiments, the drug moiety comprises an acidic, a basic, or a zwitterionic structural element; or an acidic, a basic, or a zwitterionic residual structural element paired with an ion to form a salt. The invention further relates to a composition comprising the substance and an inactive ingredient, and to a dosage form comprising the composition. The invention relates to a method of treating a disease or condition comprising: administering the substance to a patient need thereof. In preferable embodiments, the substance is administered via an oral, intravenous, subcutaneous, intramuscular, transdermal, intraarterial, intraarticular, or intradermal route.

The invention relates to a method of making a composition comprising: providing a drug moiety in an ionic form; providing a transport moiety in an ionic form; combining the drug moiety and the transport moiety, in the presence of a solvent having a dielectric constant less than that of water, to form a complex; and separating the complex from the solvent. In preferable embodiments, the transport moiety comprises an acidic, a basic, or a zwitterionic structural element; or an acidic, a basic, or a zwitterionic residual structural element paired with an ion in the transport moiety. In preferable embodiments, the drug moiety comprises an acidic, a basic, or a zwitterionic structural element; or an acidic, a basic, or a zwitterionic residual structural element paired with an ion in the transport moiety. In preferable embodiments, the drug moiety comprises an acidic structural element or an acidic residual structural element; and the drug moiety is processed to obtain the acid form of the drug moiety. In preferable embodiments, the drug moiety comprises a basic structural element or a basic residual structural element; and the drug moiety is processed to obtain the basic form of the drug moiety. In preferable embodiments, the drug moiety comprises a zwitterionic structural element or zwitterionic residual structural element; and a non-bonding structural element or residual structural element of the zwitterionic structural element or zwitterionic residual structural element is blocked before reacting the drug moiety and the transport moiety.

The invention relates to a method of treatment comprising: providing a drug moiety in an ionic form; providing a transport moiety in an ionic form; combining the drug moiety and the transport moiety, in the presence of a solvent having a dielectric constant less than that of water, to form a complex; separating the complex from the solvent; and administering the separated complex to a patient in need thereof. In preferable embodiments, the transport moiety comprises an acidic, a basic, or a zwitterionic structural element; or an acidic, a basic, or a zwitterionic residual structural element paired with an ion to form a salt. In preferable embodiments, the drug moiety comprises an acidic, a basic, or a zwitterionic structural element; or an acidic, a basic, or a zwitterionic residual structural element paired with an ion to form a salt. In preferable embodiments, the drug moiety comprises an acidic structural element or an acidic residual structural element; and the drug moiety is processed to obtain the acid form of the drug moiety. In preferable embodiments, the drug moiety comprises a basic structural element or a basic residual structural element; and the drug moiety is processed to obtain the basic form of the drug moiety. In preferable embodiments, the drug moiety comprises a zwitterionic structural element or zwitterionic residual structural element; and a non-bonding structural element or residual structural element of the zwitterionic structural element or zwitterionic residual structural element is blocked before reacting the drug moiety and the transport moiety. In preferable embodiments, the complex is administered via an oral, intravenous, subcutaneous, intramuscular, transdermal, intraarterial, intraarticular, or intradermal route.

The invention additionally relates to a method of improving absorption of a drug moiety comprising: providing a complex of the drug moiety and a transport moiety; and administering the complex to a patient in need thereof. In preferable embodiments, the complex is administered orally, and the improved absorption comprises improved oral absorption. In preferable embodiments, the improved oral absorption comprises improved lower gastrointestinal tract absorption. In preferable embodiments, the improved oral absorption comprises improved upper gastrointestinal tract absorption. In preferable embodiments, the complex is administered transdermally, and the improved absorption is improved transdermal absorption. In preferable embodiments, the complex is administered subcutaneously, and the improved absorption is improved subcutaneous absorption.

While there has been described and pointed out features and advantages of the invention, as applied to present embodiments, those skilled in the medical art will appreciate that various modifications, changes, additions, and omissions in the method described in the specification can be made without departing from the spirit of the invention.

### EXAMPLES

The following examples are meant to be illustrative of the claimed invention and not limiting in any manner.

### Metformin

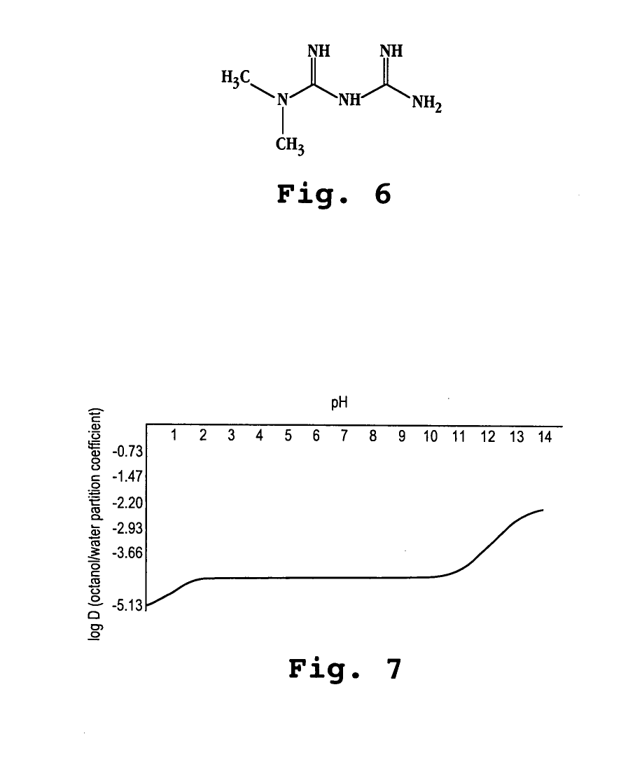

Metformin refers to N,N-dimethylimidodicarbonimidic diamide, and has a molecular formula of C₄H₁₁N₅, molecular weight of 129.17. The compound is commercially available as metformin hydrochloride. FIG. 6 shows the chemical structure of metformin.

### Example 1

### Preparation of Metformin-Transport Moiety Complex

Materials:

| | | |
|---|---|---|
| metformin hydrochloride | 13.0 | g |
| lauric acid | 16.0 | g |
| methanol | 675 | mL |
| acetone | 300 | mL |
| demineralized water | 14 | mL |
| anionic resin (Amberlyst A-26 (OH)) | 108 | g |

Preparation of Metformin Base

The ion exchange column was packed with the anionic resin, Amberlyst A-26 (OH) and a net weight was obtained.

The column was rinsed first with deionized (DI) water (backflush) and then rinsed with methanol containing 2% v/v DI water, with care taken to not allow the column to dry out.

Metformin hydrochloride was dissolved in an eluant comprised of 365 mL methanol containing 2% DI water by volume.

The solution of step 3 was passed through the column dropwise using a separatory funnel and the eluate collected. The total metformin hydrochloride passed through was calculated to be less than the ion exchange resin's equilibrating point (capacity). The column was rinsed with approximately an equal volume of eluant. A total of 690 mL of eluate of the metformin base was collected.

The combined eluates were evaporated to dryness under vacuum at an external temperature of 40° C., raised to 65° C. at the end of the concentration step to remove all the remaining water. This concentration step was carried out in the most expeditious manner due to the instability of the metformin base.

Complex Formation

A lauric acid-acetone solution, 16.0 g lauric acid dissolved in 300 mL acetone, was prepared. The concentrated metformin base from step 5 above was dissolved using several washings of acetone and these washings were immediately filtered in the presence of filter-aid to remove any unconverted metformin hydrochloride. The filtrate was collected in an Erlenmeyer flask and, with stirring, the lauric acid-acetone solution was added at a fast drop, using a separatory funnel.

Metformin laurate precipitated out. Stirring was continued overnight at ambient temperature (20-25° C.).

The mixture of solvent and precipitated metformin laurate was filtered through a Buckner funnel. The filter cake was rinsed with 4×200 mL acetone and then dried under vacuum suction for an hour. The filter cake was scraped off the filter paper and weighed. The melting point was determined in a capillary tube. Final drying was in a vacuum oven for 3 hours at ambient temperature was done.

The above procedure resulted in formation of a complex of metformin laurate with a melting point of 150°-153 ° C. The melting point of metformin hydrochloride is reported as 225° C. Total Yield=75% relative to theoretical amount calculated from the stoichiometric amounts of metformin hydrochloride and lauric acid used.

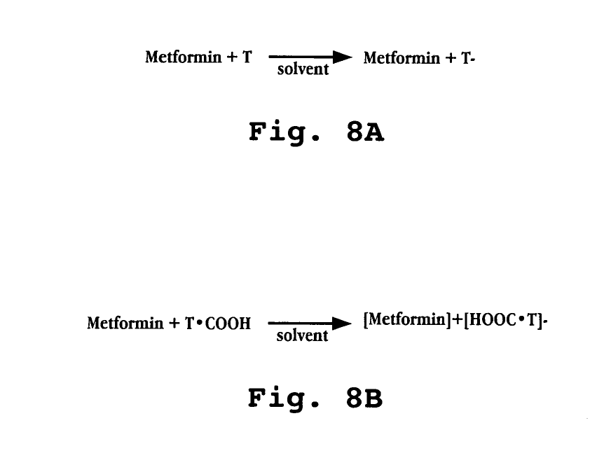

FIG. 8A shows a generalized synthetic reaction scheme for preparation of a metformin-transport moiety complex. FIG. 8B shows a generalized synthetic reaction scheme for preparation of a metformin-transport moiety complex, where the transport moiety includes a carboxyl group. FIG. 8C shows a synthetic reaction scheme for preparation of a metformin-fatty acid complex, as illustrated in this Example.

### Example 2

Characterization of Metformin-Transport Moiety Complex

HPLC Characterization

Reverse phase high pressure liquid chromatography (RP-HPLC) was used to analyze the metformin-laurate complex formed as described in Example 1. For comparison, HPLC traces of metformin HCl, of sodium laurate, and of a physical mixture of metformin HCl and sodium laurate were also generated. Reverse phase was conducted on a Hewlett Packard 1100 liquid chromatograph with an evaporative light scattering detector and using a C3 column (Agilest Zorbas SB C3, 5 μm, 3.0×75 mm. A mobile phase of water:acetonitrile 50:50 v:v was used. Column temperature was 40° C. and the flow rate was 0.5 mL/min.

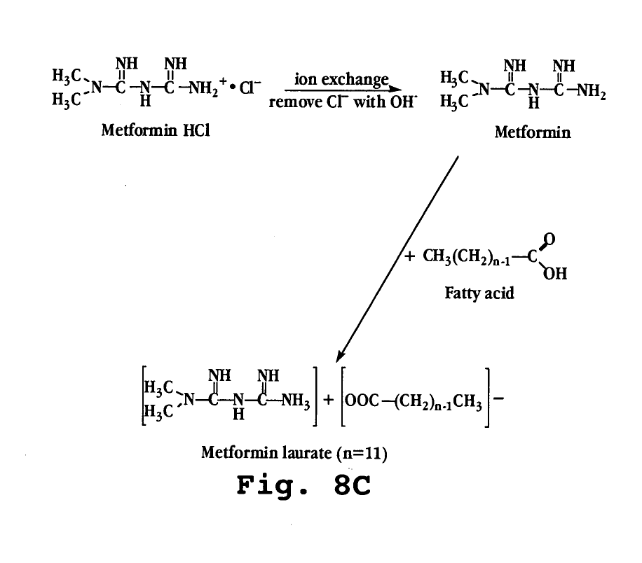

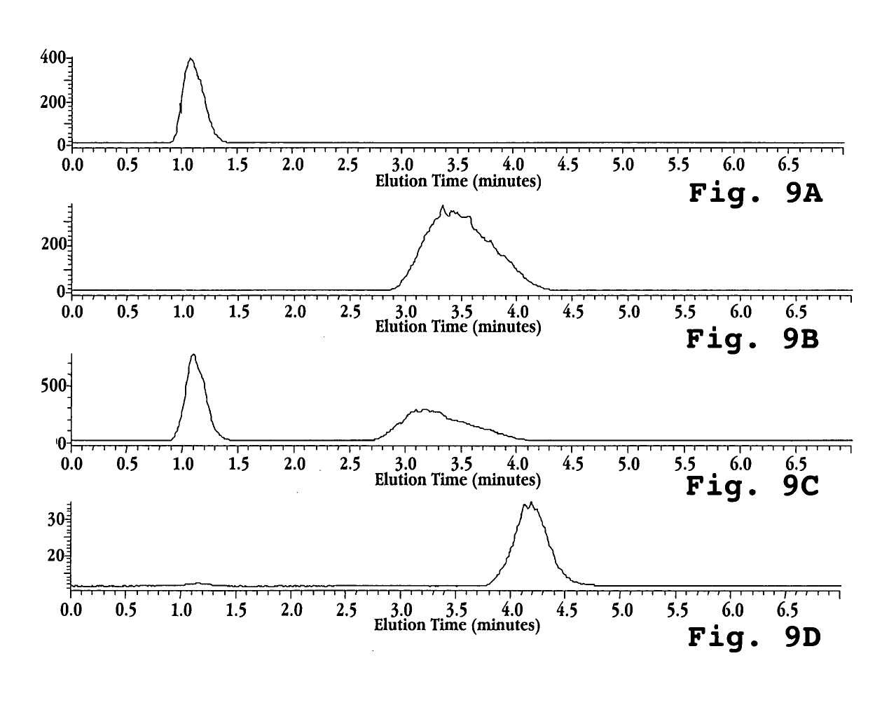

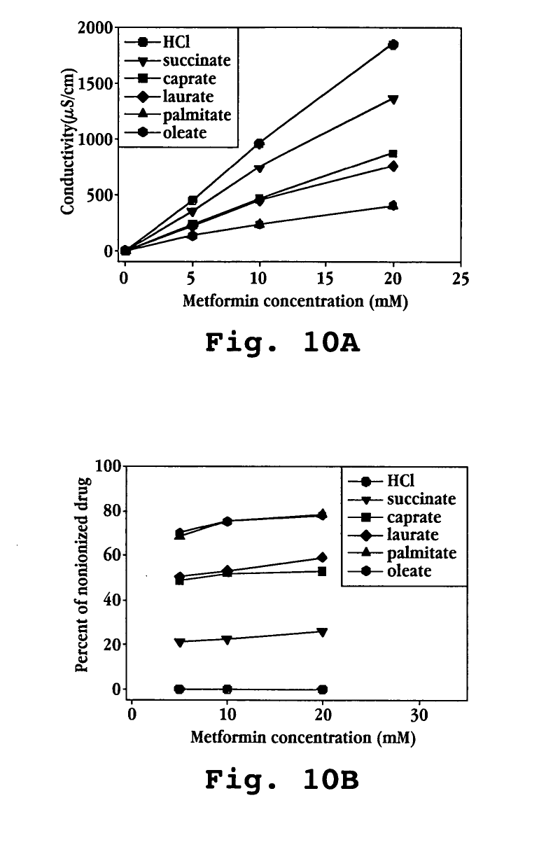

The results are shown in FIGS. 9A-9D. The trace for metformin hydrochloride is shown in FIG. 9A, and a single peak at 1.1 minutes is observed. The salt form of lauric acid, sodium laurate, elutes as a single, broad peak between about 3-4 minutes (FIG. 9B). A 1:1 molar mixture of metformin HCl and sodium laurate in water elutes as two peaks, one peak at 1.1 minutes corresponding to metformin hydrochloride and a second peak between about 2.7-4 minutes of sodium laurate (FIG. 9C). FIG. 9D shows the HPLC trace for the complex formed by the procedure in Example 1, where a single peak eluting between 3.9-4.5 minutes is observed. The HPLC traces show that the complex formed of metformin base and lauric acid is different from the physical mixture of the two components in water. The trace also shows that the complex does not dissociate when subjected to the solvent system (water:acetonitrile 50:50 v:v) for the HPLC analysis.

Octanol/Water Partition Coefficient

In another study to characterize the metformin-laurate complex, the octanol/water partition coefficient (D=C_{octanol}/C_{water}) of the complex was measured and compared to metformin HCl, a 1:1 (mol/mol) mixture of metformin hydrochloride:sodium lauryl sulfate and 1:1 (mol/mol) mixture of metformin hydrochloride:sodium laurate. The results are shown in Table 3.

TABLE 3
OCTANOL/WATER PARTITION COEFFICENTS

| Test Species | *LogD |
|---|---|
| Metformin HCl | −2.64 |
| 1:1, Metformin HCl:Sodium lauryl sulfate | −0.05 |
| 1:1, Metformin HCl:Sodium laurate | 0.06 |
| Metformin laurate | 0.44 |

*Log[C_{octanol}/C_{water}]

The complex had a log D of 0.44, a significant increase compared to metformin hydrochloride, indicating that the complex partitions more favorably in to octanol than does the salt form of metformin. The complex also had a higher log D compared to the physical mixtures of metformin hydrochloride in the fatty acid salts. This difference in log D further confirms that the complex of metformin-fatty acid is not a physical mixture of the two species, i.e., a simple ion pair, but is of another form. FIG. 7 is a plot of the logarithm of the octanol/water partition coefficient as a function of pH for metformin HCl.

Dissociation Properties

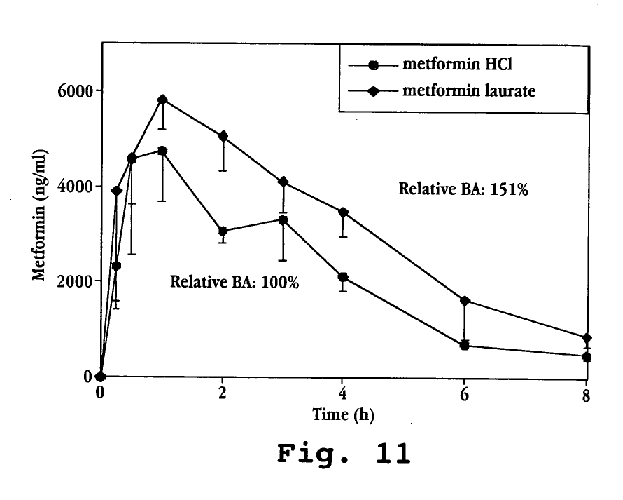

Metformin-fatty acid complexes were prepared according to the procedure described in Example 1 using the fatty acids capric acid, lauric acid, palmitic acid, and oleic acid. A complex of metformin and succinic acid was also prepared. The complexes were characterized by melting points and solubility and the data is summarized in Table 4A. Additionally, the conductivity of the various complexes in aqueous solutions (pH≅5.8) was measured with a CDM 83 conductivity meter (Radiometer Copenhagen) at 23 ° C. The values are summarized in Table 4B and presented graphically in FIG. 10A.

TABLE 4A

| Metformin Salt or | Melting Point | H₂O Solubility |
|---|---|---|
| Complex | (° C.) | (4 h) |
| HCI | 238 | >300 |
| succinate | 243 | 95 |
| palmitate | 150 | 12 |
| Oleate | 138 | 53 |
| Caprate | 153 | gelation |
| Laurate | 151 | gelation |

TABLE 4B

| | Conductivity (μS/cm) | | | | | |
|---|---|---|---|---|---|---|
| Metformin | Metformin | Metformin | Metformin | Metformin | Metformin | Metformin |
| Concentrate | HCI | succinate | caprate | laurate | palmitate | oleate |
| 20 (mM) | 1850 | 1370 | 872 | 7580 | 398 | 405 |
| 10 (mM) | 958 | 741 | 461 | 450 | 237 | 235 |
| 5 (mM) | 452 | 355 | 233 | 225 | 144 | 136 |
| 0 (mM) | 1.26 | 1.26 | 1.26 | 1.26 | 1.26 | 1.26 |

FIG. 10A shows the conductivity, in microsiemens/centimeter (μS/cm) as a function of metformin concentration for metformin HCl (circles), metformin complexed with succinate (inverted triangles), caprate (squares), laurate (diamonds), palmitate (triangles), and oleate (octagons). Metformin HCl had the highest conductivity at all concentrations. The complexes had a lower conductivity than metformin hydrochloride, with a decreasing conductivity with increasing fatty acid carbon number apparent.

FIG. 10B shows the percent of non-ionized drug for each of the complexes as a function of metformin concentration, determined from Equation 3. Metformin HCl (circles) is completely ionized, whereas metformin-succinate (inverted triangles) is about 80% ionized. The complexes metformin-caprate (squares) and metformin-laurate (diamonds) and about 50% ionized, and metformin-palmitate (triangles), and metformin-oleate (octagons) are about 30% ionized. Again this data establishes a difference between the ion pair metformin hydrochloride and the metformin-fatty acid complexes.

### Example 3

In Vivo Lower G.I. Tract Absorption Using Oral Gavage Rat Model

Eight rats were randomized into two treatment groups. After being fasted for 12-24 hours, the first group was given by oral gavage 40 mg/kg free base equivalent of metformin hydrochloride. The second group received by oral gavage 40 mg/kg free base equivalent of metformin laurate complex, prepared as described in Example 1. Blood samples were taken from the tail vein 15 minutes, 30 minutes, 1 hour, 1.5 hours, 2 hours, 3 hours, 4 hours, 6 hours, and 8 hours after oral gavage. The metformin plasma concentration was analyzed by LC/MS/MS.

The results are shown in FIG. 11. The plasma concentration in rats given metformin HCl (circles) by oral gavage reached a plasma concentration maximum about 1 hour after treatment, with a Cmax of about 4080 ng/mL. Rats treated by oral gavage with the metformin-laurate complex (diamonds) had a plasma concentration maximum about 1 hour after treatment, with a Cmax of about 5090 ng/mL. The plasma concentration for rats treated with the complex was higher at all test points in the period 1-8 hours after treatment. Analysis of the data showed that the relative bioavailability of metformin when administered in the form of the complex was 151%, relative to the bioavailability of metformin when administered as metformin HCl (100% bioavailability).

At the end of the study, the rats were euthanized and a macroscopic evaluation of the G.I. tract of the test animals was conducted to look for signs of irritation. No irritation in the rats treated with the complex or with metformin HCl was observed.

### Example 4

In Vivo Absorption Using Flushed Ligated Colonic Model in Rats

An animal model commonly known as the “intracolonic ligated model” was employed for testing formulations. Surgical preparation of a fasted anesthetized 0.3-0.5 kg Sprague-Dawley male rats proceeded as follows. A segment of proximal colon was isolated and the colon was flushed of fecal materials. The segment was ligated at both ends while a catheter was placed in the lumen and exteriorized above the skin for delivery of test formulation. The colonic contents were flushed out and the colon was returned to the abdomen of the animal. Depending on the experimental set up, the test formulation was added after the segment was filled with 1 mL/kg of 20 mM sodium phosphate buffer, pH 7.4, to more accurately simulate the actual colon environment in a clinical situation.

Rats were allowed to equilibrate for approximately 1 hour after surgical preparation and prior to exposure to each test formulation. Metformin HCl or a metformin-fatty acid complex were administered as an intracolonic bolus at dosages of 10 mg metformin HCl/rat or 10 mg metformin complex/rat. Rats were treated with metformin-fatty acid complexes prepared as described in Example 1, with the fatty acids capric acid, lauric acid, palmitic acid, and oleic acid, and with a succinate acid dimer. Blood samples were obtained from the jugular catheter at 0, 15, 30, 60, 90, 120, 180 and 240 minutes after administration of the test formulation and analyzed for blood metformin concentration. Tables 5-10 below show for each complex and for each rat the concentration of metformin base detected in the blood plasma measured in nanograms per milliliter at each time point.

TABLE 5
Metformin HCl

| Time | Rat1 | Rat2 | Rat3 | | Standard |
|---|---|---|---|---|---|
| (h) | (ng/ml) | (ng/ml) | (ng/ml) | Average | Deviation |
| 0 | 0 | 0 | 0 | 0.0 | 0.0 |
| 0.25 | 48.9 | 30.7 | 52.3 | 44.0 | 11.6 |
| 0.5 | 48.4 | 22.2 | 61.2 | 43.9 | 19.9 |
| 1 | 16.5 | 56.6 | 67.5 | 46.9 | 26.9 |
| 1.5 | 14 | 79 | 99 | 64.0 | 44.4 |
| 2 | 27.3 | 124 | 96.6 | 82.6 | 49.8 |
| 3 | 28.6 | | 81 | 54.8 | 37.1 |
| 4 | 28.2 | | 83.9 | 56.1 | 39.4 |

TABLE 6
Metformin Succinate

| Time | Rat1 | Rat2 | Rat3 | | Standard |
|---|---|---|---|---|---|
| (h) | (ng/ml) | (ng/ml) | (ng/ml) | Average | Deviation |
| 0 | 0 | 0 | 0 | 0.0 | 0.0 |
| 0.25 | 19.9 | 42.6 | 19 | 27.2 | 13.4 |
| 0.5 | 19.5 | 53.6 | 18.9 | 30.7 | 19.9 |
| 1 | 24.3 | 89.8 | 19 | 44.4 | 39.4 |
| 1.5 | 26.2 | 78.4 | 15.7 | 40.1 | 33.6 |
| 2 | 44.1 | 59.1 | 13.2 | 38.8 | 23.4 |
| 3 | 36.9 | 48.3 | 15.8 | 33.7 | 16.5 |
| 4 | 62.4 | 60.4 | 19 | 47.3 | 24.5 |

TABLE 7
Metformin Palmitate

| Time | Rat1 | Rat2 | Rat3 | | Standard |
|---|---|---|---|---|---|
| (h) | (ng/ml) | (ng/ml) | (ng/ml) | Average | Deviation |
| 0 | 0 | 0 | 0 | 0.0 | 0.0 |
| 0.25 | 535 | 126 | 233 | 298.0 | 212.1 |
| 0.5 | 393 | 145 | 245 | 261.0 | 124.8 |
| 1 | 147 | 43.6 | 212 | 134.2 | 84.9 |
| 1.5 | 103 | 116 | 110 | 109.7 | 6.5 |
| 2 | 86.3 | 115 | 92.5 | 97.9 | 15.1 |
| 3 | 52 | 77.7 | 76.6 | 68.8 | 14.5 |
| 4 | 57.9 | 105 | 118 | 93.6 | 31.6 |

TABLE 8
Metformin Oleate

| Time | Rat1 | Rat2 | Rat3 | | Standard |
|---|---|---|---|---|---|
| (h) | (ng/ml) | (ng/ml) | (ng/ml) | Average | Deviation |
| 0 | 0 | 0 | 0 | 0.0 | 0.0 |
| 0.25 | 262 | 788 | 2450 | 1166.7 | 1142.1 |
| 0.5 | 218 | 552 | 1350 | 706.7 | 581.6 |
| 1 | 79.3 | 426 | 1040 | 515.1 | 486.5 |
| 1.5 | 87.3 | 342 | 626 | 351.8 | 269.5 |
| 2 | 59.3 | 399 | 219 | 225.8 | 170.0 |
| 3 | 27.9 | 163 | 79.3 | 90.1 | 68.2 |
| 4 | 30.2 | 122 | 37.3 | 63.2 | 51.1 |

TABLE 9
Metformin Caprate

| Time | Rat1 | Rat2 | Rat3 | | Standard |
|---|---|---|---|---|---|
| (h) | (ng/ml) | (ng/ml) | (ng/ml) | Average | Deviation |
| 0 | 0 | 0 | 0 | 0.0 | 0.0 |
| 0.25 | 2750 | 3390 | 5020 | 3720.0 | 1170.4 |
| 0.5 | 1850 | 1430 | 2040 | 1773.3 | 312.1 |
| 1 | 613 | 447 | 876 | 645.3 | 216.3 |
| 1.5 | 264 | 222 | 502 | 329.3 | 151.0 |
| 2 | 177 | 243 | 193 | 204.3 | 34.4 |
| 3 | 51.8 | 97.9 | 100 | 83.2 | 27.2 |
| 4 | 40.9 | 68 | 66.7 | 58.5 | 15.3 |

TABLE 10
Metformin Laurate

| Time | Rat1 | Rat2 | Rat3 | | Standard |
|---|---|---|---|---|---|
| (h) | (ng/ml) | (ng/ml) | (ng/ml) | Average | Deviation |
| 0 | 0 | 0 | 0 | 0.0 | 0.0 |
| 0.25 | 3570 | 3270 | 3280 | 3373.3 | 170.4 |
| 0.5 | 2060 | 1950 | 1100 | 1703.3 | 525.4 |
| 1 | 960 | 1590 | 544 | 1031.3 | 526.6 |
| 1.5 | 494 | 1150 | 287 | 643.7 | 450.5 |
| 2 | 237 | 378 | 112 | 242.3 | 133.1 |
| 3 | 102 | 171 | 58.3 | 110.4 | 56.8 |
| 4 | 68.1 | 77.5 | 47.4 | 64.3 | 15.4 |

For comparison, metformin HCl, in a dosage of 2 mg/kg of rat body weight was injected intravenously directly into the blood stream of three test rats. Blood samples were taken periodically over a four hour period for analysis of metformin base. The results are shown in Table 11.

TABLE 11
Metformin HCl (iv)

| Time | Rat1 | Rat2 | Rat3 | | Standard |
|---|---|---|---|---|---|
| (h) | (ng/ml) | (ng/ml) | (ng/ml) | Average | Deviation |
| 0 | 0 | 0 | 0 | 0.0 | 0.0 |
| 0.033333 | 1430 | 2370 | 3650 | 2483.3 | 1114.3 |
| 0.25 | 133 | 545 | 276 | 318.0 | 209.2 |
| 0.5 | 262 | 331 | 94.7 | 229.2 | 121.5 |
| 1 | 35.5 | N/A | 25.2 | 30.4 | 7.3 |
| 1.5 | 0 | 66.3 | 0 | 22.1 | 38.3 |
| 2 | 0 | 0 | 0 | 0.0 | 0.0 |
| 3 | 0 | 0 | 0 | 0.0 | 0.0 |

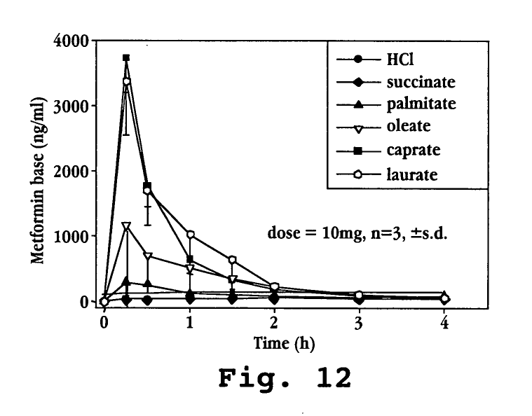

The results in Tables 5-10 are shown in graphically in FIG. 12. FIG. 12 shows the metformin plasma concentration, in ng/mL, in rats as a function of time, in hours, for metformin HCl (circles), metformin complexed with succinate (diamonds), palmitate (triangles), oleate (inverted triangles), caprate (squares), and laurate (octagons). The highest blood plasma concentrations were obtained from the complexes prepared from lauric acid (circles) and with capric acid (squares). Complexes with palmitic acid (triangles) and oleic acid (inverted triangles) achieved metformin plasma concentrations lower than that achieved form the complexes with lauric acid and capric acid, but higher than the plasma concentration provided by metformin HCl or by metformin succinate.

Table 12 shows the relative Cmax (maximum plasma concentration of metformin base for each complex relative to the plasma concentration of metformin HCl), and the relative bioavailability of each complex normalized to the bioavailability of metformin HCl given via intubation to a ligated (fourth column) and relative to the bioavailability of metformin HCl given intravenously (third column).

TABLE 12

| | | Area Under | | |
|---|---|---|---|---|
| | | the Curve | Bioavailability | Bioavailability |
| Metformin | | (0-4 hrs, | Relative to | Relative |
| Test | Relative | based on 1 mg | IV Dose¹ | to IV Dose² |
| Compound | Cₘₐₓ | base/rat) | (%) | (Fold-Increase) |
| HCl (i.v.) | 1.0 | 692.5 | 100.0 | — |
| HCl | | 29.2 | 4.2 | 1 |
| succinate | 0.6 | 21.4 | 3.1 | 0.7 |
| palmitate | 3.6 | 144.3 | 20.8 | 5 |
| Oleate | 14.1 | 408.3 | 59.0 | 14 |
| Caprate | 45.0 | 548.0 | 79.1 | 19 |
| Laurate | 40.8 | 674.5 | 97.4 | 23.0 |

¹AUC achieved by each complex normalized to the AUC of metformin HCl given intravenously; (ng · h/mL-mg).
²AUC achieved by each complex normalized to the AUC of metformin HCl given via intubation to ligated colon.

Metformin when provided for absorption to the lower G.I. tract in the form of a metformin-transport moiety complex is significantly enhanced, as seen by the nearly 5-fold increase in bioavailability achieved with a metformin-palmitate complex, relative to that of the HCl salt. The oleate complex yielded a 14-fold improvement in bioavailability relative to that of the HCl salt. The metformin-caprate complex provided an almost 18-fold improvement bioavailability relative to that of the HCl salt. The metformin-laurate complex yielded a greater than 20-fold improvement in bioavailability relative to that of the HCl salt. Accordingly, the invention contemplates a compound comprised of a complex formed of metformin and a transport moiety, wherein the complex provides at least a 5 fold increase, more preferably at least a 15 fold increase, and more preferably at least a 20 fold increase in lower G.I. tract absorption relative to the absorption of metformin HCl in the lower G.I. tract, as evidenced by metformin bioavailability determined from metformin plasma concentration. Thus, metformin when administered in the form of a metformin-transport moiety complex provides a significantly enhanced lower G.I. tract absorption of metformin into the blood.

### Example 5

In Vivo Absorption Using Flushed Ligated Colonic Model in Rats

Another study was conducted using the flush-ligated colonic model described in Example 4 to compare the bioavailability of metformin when provided in the form of a complex to the bioavailability of metformin when provided as a physical mixture of metformin HCl and sodium laurate (1:1 molar ratio). Various doses of the two test formulations (metformin-laurate complex and 1:1 molar ratio metformin HCl:sodium laurate) or of metformin HCl were intubated into the ligated colon. Plasma samples were analyzed for metformin concentration and bioavailability determined relative to the bioavailability of intravenously administered metformin. The results are shown in FIG. 13.

FIG. 13 shows the percent bioavailability as a function of metformin dose, in mg base/kg, of the physical mixture of metformin HCl and sodium laurate (circles) and of the metformin laurate complex (squares). The complex had a higher bioavailability with lower variability than the physical mixture.

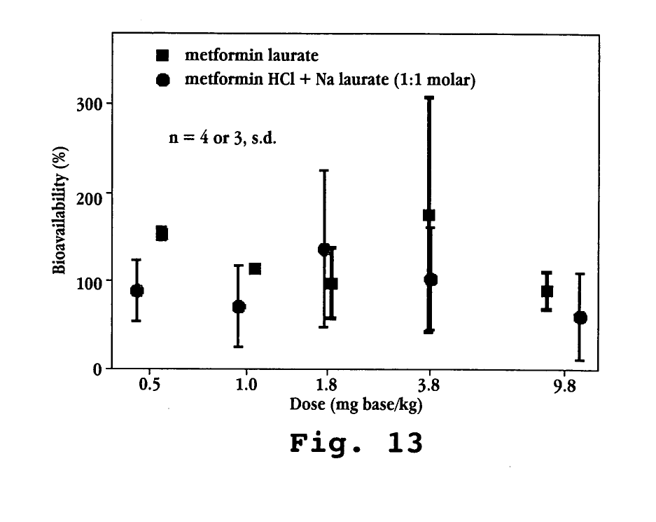

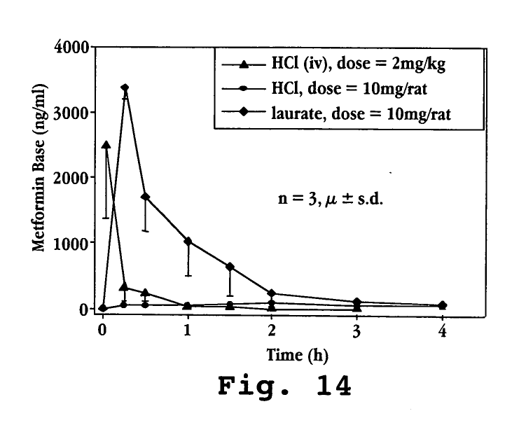

FIG. 14 shows the data from Tables 5, 11, and 12 in Example 3, to illustrate the pharmacokinetics of the complex (diamonds) compared to metformin HCl administered via intubation to the ligated colon (circles) or intravenously (triangles). The complex provides a higher colonic absorption than the salt form of the drug, and has a longer lasting blood concentration that intravenous administration.

### Example 6

Preparation of Dosage Form Comprising a Metformin-Transport Moiety Complex

A dosage form comprising a layer of metformin HCl and a layer of metformin-laurate complex was prepared as follows.

10 grams of metformin hydrochloride, 1.18 g of polyethylene oxide of 100,000 molecular weight, and 0.53 g of polyvinylpyrrolidone having molecular weight of about 38,000 were dry blended in a conventional blender for 20 minutes to yield a homogenous blend. Next, 4 mL denatured anhydrous alcohol was added slowly, with the mixer continuously blending, to the three component dry blend. The mixing was continued for another 5 to 8 minutes. The blended wet composition was passed through a 16 mesh screen and dried overnight at room temperature. Then, the dry granules were passed through a 16 mesh screen and 0.06 g of magnesium stearate were added and all the ingredients were dry blended for 5 minutes. The fresh granules were ready for formulation as the initial dosage layer in the dosage form. The granules were comprised of 85.0 wt % metformin hydrochloride, 10.0 wt % polyethylene oxide of 100,000 molecular weight, 4.5 wt % polyvinylpyrrolidone having a molecular weight of about 35,000 to 40,000, and 0.5 wt % magnesium stearate.

The metformin-laurate layer in the dosage form was prepared as follows. First, 9.30 grams of metformin laurate complex, prepared as described in Example 1, 0.50 g polyethylene oxide of 5,000,000 molecular weight, 0.10 g of polyvinylpyrrolidone having molecular weight of about 38,000 were dry blended in a conventional blender for 20 minutes to yield a homogenous blend. Next, denatured anhydrous ethanol was added slowly to the blend with continuous mixing for 5 minutes. The blended wet composition was passed through a 16 mesh screen and dried overnight at room temperature. Then, the dry granules were passed through a 16 mesh screen and 0.10 g magnesium stearate were added and all the dry ingredients were dry blended for 5 minutes. The composition was comprised of 93.0 wt % metformin laurate, 5.0 wt % polyethylene oxide 5,000,000 molecular weight, 1.0 wt % polyvinylpyrrolidone having molecular weight of about 35,000 to 40,000 and 1.0 wt % magnesium stearate.

A push layer comprised of an osmopolymer hydrogel composition was prepared as follows. First, 58.67 g of pharmaceutically acceptable polyethylene oxide comprising a 7,000,000 molecular weight, 5 g Carbopol® 974P, 30 g sodium chloride and 1 g ferric oxide were separately screened through a 40 mesh screen. The screened ingredients were mixed with 5 g of hydroxypropylmethylcellulose of 9,200 molecular weight to produce a homogenous blend. Next, 50 mL of denatured anhydrous alcohol was added slowly to the blend with continuous mixing for 5 minutes. Then, 0.080 g of butylated hydroxytoluene was added followed by more blending. The freshly prepared granulation was passed through a 20 mesh screen and allowed to dry for 20 hours at room temperature (ambient). The dried ingredients were passed through a 20 mesh screen and 0.25 g of magnesium stearate was added and all the ingredients were blended for 5 minutes. The final composition was comprised of 58.7 wt % of polyethylene oxide, 30.0 wt % sodium chloride, 5.0 wt % Carbopol®, 5.0 wt % hydroxypropylmethylcellulose, 1.0 wt % ferric oxide, 0.25 wt % magnesium stearate, and 0.08 wt % butylated hydroxytoluene.

The tri-layer dosage form was prepared as follows. First, 118 mg of the metfornin hydrochloride composition was added to a punch and die set and tamped, then 427 mg of the metfornin laurate composition was added to the die set as the second layer and again tamped. Then, 272 mg of the hydrogel composition was added and the three layers compressed under a compression force of 1.0 ton (1000 kg) into a 9/32 inch (0.714 cm) diameter punch die set, forming an intimate tri-layered core (tablet).

A semipermeable wall-forming composition was prepared comprising 80.0 wt % cellulose acetate having a 39.8% acetyl content and 20.0% polyoxyethylene-polyoxypropylene copolymer having a molecular weight of 7680-95 1 0 by dissolving the ingredients in acetone in a 80:20 wt/wt composition to make a 5.0% solids solution. Placing the solution container in a warm water bath during this step accelerated the dissolution of the components. The wall-forming composition was sprayed onto and around the tri-layered core to provide a 93 mg thickness semi-permeable wall.

Next, a 40 mil (1.02 mm) exit orifice was laser drilled in the semipermeable walled tri-layered tablet to provide contact of the metformin layer with the exterior of the delivery device. The dosage form was dried to remove any residual solvent and water.

The in vitro dissolution rates of the dosage form was determined by placing a dosage form in the metal coil sample holders attached to a USP Type VII bath indexer in a constant temperature water bath at 37° C. Aliquots of the release media were injected into a chromatographic system to quantify the amounts of drug released into a medium simulating artificial gastric fluid (AGF) during each testing interval. Three dosage forms were tested and the average dissolution rate is shown in FIG. 15B, where the release rate of metformin, in mg/hour, is shown as a function of time, in hours. Four hours after contact with an aqueous environment, the dosage form begins to release a nearly uniform amount of drug for the subsequent 12 hours, with release of drug beginning to decrease at times greater than 16 hours after contact with an aqueous environment. Release of metformin hydrochloride, present in the drug layer adjacent the exit orifice, is released initially. About 8 hours after contact with an aqueous environment, release of metformin-transport moiety complex occurs, and continues at a substantially constant rate for 8 hours longer. It will be appreciated that this dosage form is designed to release metformin hydrochloride while in transit in the upper G.I. tract, corresponding approximately to the first eight hours of transit, as indicated by the dashed bars. Metformin-transport moiety complex is released as the dosage form travels through the lower G.I. tract, approximately corresponding to times longer than about 8 hours after ingestion, as indicated by the dotted bars in FIG. 15. This design takes advantage of the increased absorption in the lower G.I. tract provided by the complex.

### Gabapentin

### Example 7

Preparation of Gabapentin-Lauryl Sulfate Complex

1. A solution of 0.5 mL 36.5% hydrochloric acid (5 mmol HCI) in 25 mL deionized water was prepared.
2. 5 mmol gabapentin (0.86 g) was added to the solution in step 1. The mixture was stirred for 10 min at room temperature. Gabapentin hydrochloride was formed.
3. 5 mmol sodium lauryl sulfate (1.4 g) was added to the aqueous solution in step 2. The mixture was stirred for 20 min at room temperature. A loose ionic pair of gabapentin and lauryl sulfate was formed.
4. 50 mL dichloromethane was added to the solution in step 3. The mixture was stirred for 2 hours at room temperature.
5. The mixture of step 4 was transferred to a separatory funnel and allowed to settle for 3 hours. Two phases were formed, a lower phase of dichloromethane and an upper phase of water.
6. The upper and lower phases in step 5 were separated. The lower dichloromethane phase was recovered and the dichloromethane was evaporated to dryness at room temperature, followed by drying in a vacuum oven for 4 hours at 40 ° C. A complex of gabapentin-lauryl sulfate (1.9 g) was obtained. Total yield was 87% relative to theoretical amount calculated from the initial amounts of gabapentin and sodium lauryl sulfate.

FIG. 16C shows a synthetic reaction scheme for preparation of a gabapentin-alkyl sulfate complex.

### Example 8

Characterization of Gabapentin-Lauryl Sulfate Complex

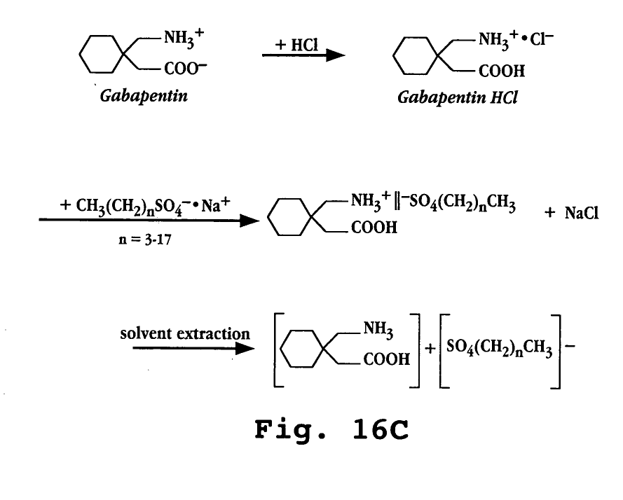

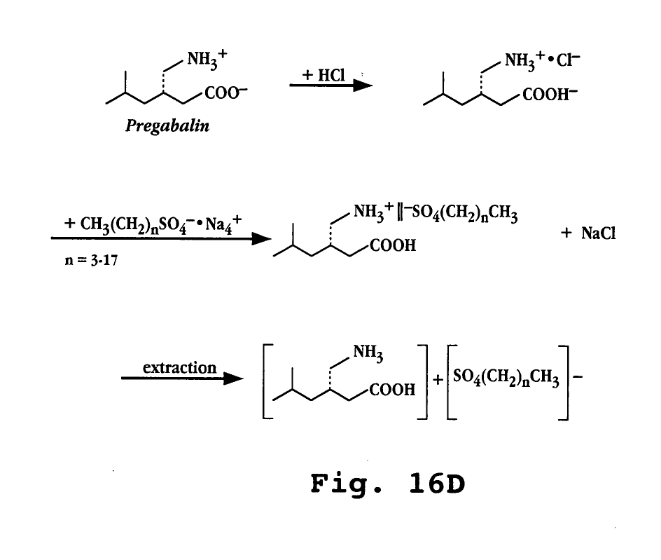

Fourier Transform Infrared Spectroscopy (FTIR) was use to analyze the gabapentin-lauryl sulfate complex formed as described in Example 7. The FTIR spectra were obtained by using a Perkin-Elmer Spectrum 2000 FTIR spectrometer system that consists of the Attenuated Total Reflectance (ATR) accessory and liquid N2 cooled MCT (mercury cadmium telluride) detector. FTIR scans of gabapentin, sodium lauryl sulfate, and of a physical mixture of gabapentin and sodium lauryl sulfate were also generated. FTIR/ATR spectra of gabapentin, sodium lauryl sulfate, and of a 1:1 molar ratio physical mixture of gabapentin and sodium lauryl sulfate (two components were dissolved in methanol and dried in air as a solid film) were also generated, and the results are shown in FIGS. 17A-17D. The spectrum for gabapentin is shown in FIG. 17A, and the peaks corresponding to the NH and COO moieties are indicated. The spectrum for sodium lauryl sulfate is shown in FIG. 17B, and a main, doublet peak corresponding to the S—O moiety is observed between 1300-1200 cm-1. A 1:1 molar mixture of gabapentin HCl and sodium lauryl sulfate in water is shown in FIG. 17C, and an attenuation of the distinct pattern characteristic of gabapentin is apparent and a broadening of the S—O peak (1300-1200 cm-1) from the sodium lauryl sulfate observed. FIG. 17D shows the FTIR spectrum for the complex formed by the procedure in Example 7, where two peaks corresponding to the COO— group of gabapentin disappeared and were replaced by a peak of COOH group in gabapentin lauryl sulfate complex, indicating the charge blocking of COO—. Deformation of N—H moiety of gabapentin was observed by the 15 cm-1 shift in the spectra of gabapentin lauryl sulfate. This shift of bands for N—H bond indicates the protonation of the N—H groups in the resulting complex. The peak at 1250 cm-1 that is indicative of the S—O absorption in the spectra of sodium lauryl sulfate was shifted 30 cm-1 as shown in the spectra of gabapentin complex, suggesting the interaction of gabapentin with sulfate group of sodium lauryl sulfate. The FTIR scans showed that the complex formed of gabapentin is different from the physical mixture of two components.

### Example 9

In Vivo Colonic Absorption Using Flushed Ligated Colonic Model in Rats

An animal model commonly known as the “flush ligated colonic model” or “intracolonic ligated model” was used. Fasted, 0.3-0.5 kg Sprague-Dawley male rats were anesthetized and a segment of proximal colon was isolated. The colon was flushed of fecal materials. The segment was ligated at both ends while a catheter was placed in the lumen and exteriorized above the skin for delivery of test formulation. The colonic contents were flushed out and the colon was returned to the abdomen of the animal. Depending on the experimental set up, the test formulation was added after the segment was filled with 1 mL/kg of 20 mM sodium phosphate buffer, pH 7.4, to more accurately simulate the actual colon environment in a clinical situation.

Rats (n=3) were allowed to equilibrate for approximately 1 hour after surgical preparation and prior to exposure to each test formulation. Gabapentin-lauryl sulfate complex or gabapentin was administered as an intracolonic bolus and delivered at 10 mg gabapentin-lauryl sulfate complex/rat or 10 mg gabapentin/rat. Blood samples obtained from the jugular catheter were taken at 0, 15, 30, 60, 90, 120, 180 and 240 minutes and analyzed for gabapentin concentration. At the end of the 4 hour test period, the rats were euthanized with an overdose of pentobarbital. Colonic segments from each rat were excised and opened longitudinally along the anti-mesenteric border. Each segment was observed macroscopically for irritation and any abnormality noted. The excised colons were placed on graph paper and measured to approximate colonic surface area. There was no histopathological change visible to the naked eye in the mucosal of any of the test rats.

A control group of rats (n=3) were treated with gabapentin intravenously, at a dose of 1 mg/rat. Blood samples were withdrawn at the same times indicated above for analysis of gabapentin concentration.

The gabapentin plasma concentration for each test animal, and the average plasma concentration for animals in each test group, are shown in Tables 13-15.

TABLE 13
Gabapentin - Intravenous Administration

| Time | Rat1 | Rat2 | Rat3 | Average | Standard |
|---|---|---|---|---|---|
| (h) | (ng/mL) | (ng/mL) | (ng/mL) | (ng/mL) | Deviation |
| 0 | 0 | 0 | 0 | 0.0 | 0.0 |
| 0.03 | 3340 | 2170 | 2330 | 2613.3 | 634.4 |
| 0.167 | 1420 | 1280 | 1080 | 1260.0 | 170.9 |
| 0.5 | 933 | 868 | 855 | 885.3 | 41.8 |
| 1 | 878 | 867 | 779 | 841.3 | 54.3 |
| 1.5 | 714 | 770 | 648 | 710.7 | 61.1 |
| 2 | 573 | 690 | 518 | 593.7 | 87.8 |
| 3 | 505 | 558 | 415 | 492.7 | 72.3 |

TABLE 14
Gabapentin - Colonic Intubation

| Time | Rat1 | Rat2 | Rat3 | Average | Standard |
|---|---|---|---|---|---|
| (h) | (ng/mL) | (ng/mL) | (ng/mL) | (ng/mL) | Deviation |
| 0 | 0 | 0 | 0 | 0.0 | 0.0 |
| 0.25 | 40.6 | 53.8 | 32 | 42.1 | 11.0 |
| 0.5 | 82.5 | 100 | 64.8 | 82.4 | 17.6 |
| 1 | 189 | 210 | 83.8 | 160.9 | 67.6 |
| 1.5 | 266 | 240 | 78.6 | 194.9 | 101.5 |
| 3 | 413 | 265 | 92.9 | 257.0 | 160.2 |
| 4 | 279 | 322 | 94.7 | 231.9 | 120.7 |

TABLE 15
Gabapentin Lauryl Sulfate - Colonic Intubation

| Time | Rat1 | Rat2 | Rat3 | Average | Standard |
|---|---|---|---|---|---|
| (h) | (ng/mL) | (ng/mL) | (ng/mL) | (ng/mL) | Deviation |
| 0 | 0 | 0 | 0 | 0.0 | 0.0 |
| 0.25 | 2160 | 2380 | 2790 | 2443.3 | 319.7 |
| 0.5 | 2110 | 2710 | 4440 | 3086.7 | 1209.8 |
| 1 | 2990 | 3280 | 3960 | 3410.0 | 497.9 |
| 1.5 | 3050 | 3270 | 3750 | 3356.7 | 358.0 |
| 3 | 2170 | 2410 | 2140 | 2240.0 | 148.0 |
| 4 | 1380 | 1520 | 1380 | 1426.7 | 80.8 |

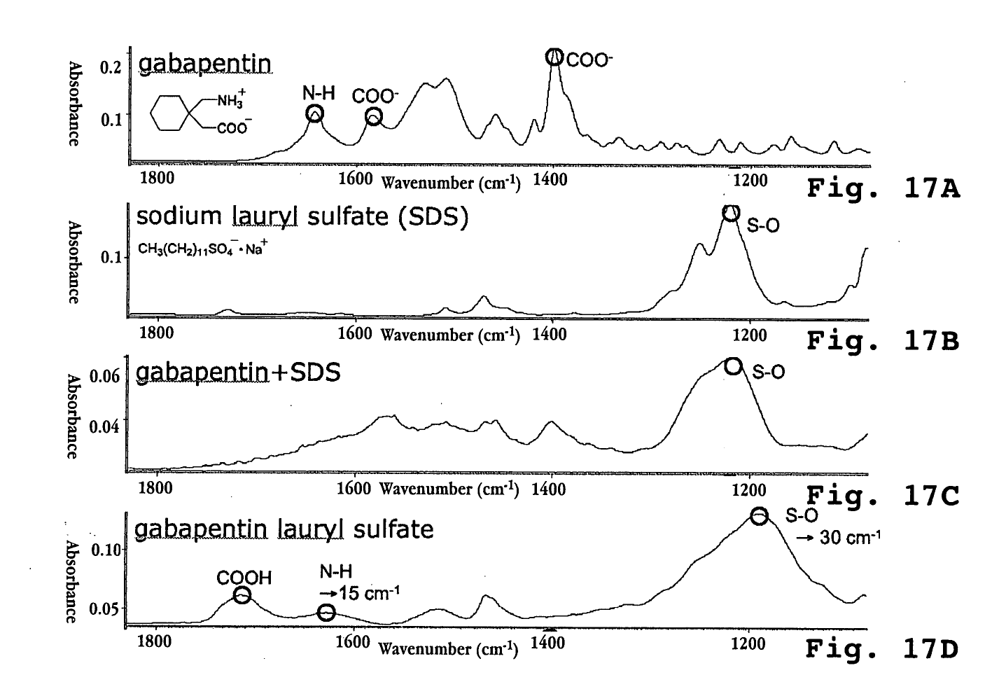

FIG. 18 shows the average gabapentin concentration in each test group as a function of time. Gabapentin administered intravenously (triangles) gives a high initial plasma concentration with a sharply decreasing concentration over the first 15 minutes. When gabapentin is administered as an intracolonic bolus (circles) a slow absorption of the drug occurs. In contract, when the drug is administered to the lower G.I. tract in the form of a gabapentin-lauryl sulfate complex (diamonds), a rapid uptake of drug occurs, with a Cmax observed one hour after intubation.

Pharmacokinetic parameters from this study are shown in Table 16. The area under the curve (AUC) is determined from time zero to time infinity based on 1 mg of gabapentin/rat for each of the gabapentin dosages, where time infinity was estimated by assuming a log-linear decline. Gabapentin bioavailability is expressed as a percent of the gabapentin concentration resulting from intravenous administration of the drug.

TABLE 16

| Drug Form | AUC∞ | bioavailability |
|---|---|---|
| (route of administration) | (ng · h/mL-mg) | (%) |
| gabapentin (iv) | 6090.3 | 100 |
| gabapentin (colonic) | 301.4 | 4.9 |
| gabapentin lauryl | 3854.1 | 63.3 |
| sulfate complex (colonic) | | |

The enhanced colonic absorption provided by the complex of gabapentin and lauryl sulfate is apparent from the markedly improved bioavailability of the drug when administered to the lower G.I. tract in the form of the complex relative to the neat drug. The gabapentin-lauryl sulfate complex provided a 13-fold improvement in bioavailability relative to that of the neat drug. Accordingly, the invention contemplates a compound comprised of, consisting essentially of, or consisting of a complex formed of gabapentin (or pregabalin) and a transport moiety, wherein the complex provides at least a 5 fold increase, more preferably at least a 10 fold increase, and more preferably at least a 12-fold increase in colonic absorption relative to colonic absorption of gabapentin (or pregabalin), as evidenced by gabapentin (or pregabalin) bioavailability determined from gabapentin (or pregabalin) plasma concentration. Thus, gabapentin (or pregabalin) when administered in the form of a gabapentin (or pregabalin)-transport moiety complex provides a significantly enhanced colonic absorption of gabapentin (or pregabalin) into the blood.

### Example 10

In Vivo Absorption

Twenty-eight rats were randomized into seven test groups (n=4). Gabapentin or gabapentin-lauryl sulfate complex, prepared as described in Example 1A, was intubated via catheter into the beginning of the duodenum of rats at dosages of 5 mg/rat, 10 mg/rat, and 20 mg/rat. The remaining test group was given 1 mg/kg gabapentin intravenously.

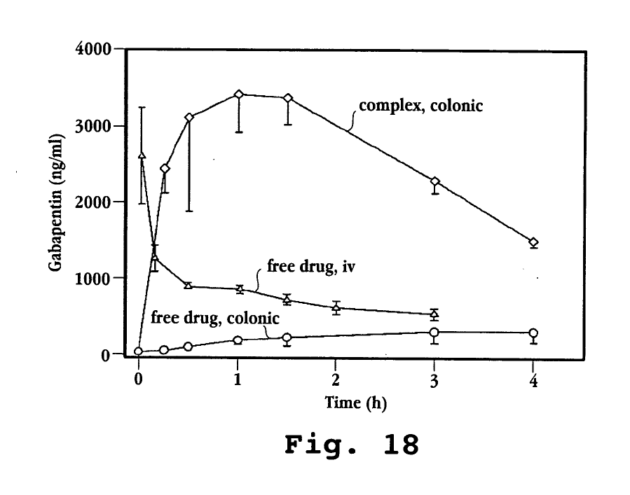

Blood samples were taken from each animal over a four hour period and analyzed for gabapentin content. The results are shown in Tables 17-22 and in FIGS. 19A-19C.

TABLE 17
Gabapentin lauryl sulfate, duodenal dose 5 mg/rat

| Time | rat1 | rat2 | rat3 | rat4 | | |
|---|---|---|---|---|---|---|
| (h) | (ng/mL) | (ng/mL) | (ng/mL) | (ng/mL) | Average | Std Dev. |
| 0 | 0 | 0 | 0 | 0 | 0 | 0 |
| 0.25 | 1490 | 1410 | 2130 | 2400 | 1857.5 | 484.4 |
| 0.5 | 2690 | 2080 | 3210 | 3700 | 2920 | 695.5 |
| 1 | 2380 | 2720 | 2750 | 4640 | 3122.5 | 1025.5 |
| 1.5 | 2500 | 2620 | 2470 | 4010 | 2900.0 | 742.8 |
| 2 | 1970 | 2740 | 1520 | 3620 | 2462.5 | 921.5 |
| 3 | 1580 | 1670 | 1230 | 2860 | 1835.0 | 709.2 |
| 4 | 967 | 1120 | 696 | 1710 | 1123.25 | 428.8 |

TABLE 18
Gabapentin lauryl sulfate, duodenal dose 10 mg/rat

| Time | rat1 | rat2 | rat3 | rat4 | | |
|---|---|---|---|---|---|---|
| (h) | (ng/mL) | (ng/mL) | (ng/mL) | (ng/mL) | Average | Std Dev. |
| 0 | 0 | 0 | 0 | 0 | 0 | 0 |
| 0.25 | 2260 | 2510 | 2440 | 3080 | 2572.5 | 354.3 |
| 0.5 | 3210 | 4010 | 3220 | 4350 | 3697.5 | 574.2 |
| 1 | 3670 | 3150 | 4010 | 4910 | 3935 | 740.0 |
| 1.5 | 2890 | 4590 | 4240 | 6370 | 4522.5 | 1433.3 |
| 2 | 2310 | 3880 | 4200 | 5190 | 3895 | 1194.8 |
| 3 | 1410 | 3630 | 5210 | 3400 | 3412.5 | 1558.7 |
| 4 | 981 | 2230 | 2430 | 1760 | 1850.2 | 644.0 |

TABLE 19
Gabapentin lauryl sulfate, duodenal dose 20 mg/rat

| Time | rat1 | rat2 | rat3 | rat4 | | |
|---|---|---|---|---|---|---|
| (h) | (ng/mL) | (ng/mL) | (ng/mL) | (ng/mL) | Average | Std Dev. |
| 0 | 0 | 0 | 0 | 0 | 0 | 0 |
| 0.25 | 5570 | 4270 | 5910 | 3420 | 4792.5 | 1156.2 |
| 0.5 | 5320 | 4680 | 6410 | 4820 | 5307.5 | 784.7 |
| 1 | 7370 | 6610 | 7000 | 6550 | 6882.5 | 381.4 |
| 1.5 | 6770 | 6820 | 7830 | 8380 | 7450 | 789.3 |
| 2 | 5670 | 6980 | 8100 | 9410 | 7540 | 1593.842 |
| 3 | 3720 | 5970 | 5880 | 7210 | 5695 | 1449.793 |
| 4 | 2570 | 4980 | 3330 | 4060 | 3735 | 1029.061 |

TABLE 20
Gabapentin, duodenal dose 5 mg/rat

| Time | rat1 | rat2 | rat3 | rat4 | | |
|---|---|---|---|---|---|---|
| (h) | (ng/mL) | (ng/mL) | (ng/mL) | (ng/mL) | Average | Std Dev. |
| 0 | 0 | 0 | 5.71 | 0 | 1.4275 | 2.855 |
| 0.25 | 3920 | 2590 | 3110 | 4020 | 3410 | 681.8 |
| 0.5 | 7500 | 4420 | 4400 | 6850 | 5792.5 | 1618.3 |
| 1 | 10800 | 7610 | 6350 | 7870 | 8157.5 | 1882.6 |
| 1.5 | 11400 | 8410 | 7260 | 7740 | 8702.5 | 1859.2 |
| 2 | 9390 | 6800 | 9370 | 6670 | 8057.5 | 1528.0 |
| 3 | 6350 | 5830 | 5640 | 5370 | 5797.5 | 413.9 |
| 4 | 4710 | 3490 | 3900 | 3350 | 3862.5 | 611.3 |

TABLE 21
Gabapentin, duodenal dose 10 mg/rat

| Time | rat1 | rat2 | rat3 | rat4 | | |
|---|---|---|---|---|---|---|
| (h) | (ng/ml) | (ng/ml) | (ng/ml) | (ng/ml) | Average | Std Dev. |
| 0 | 0 | 0 | 5.62 | 0 | 1.405 | 2.81 |
| 0.25 | 5690 | 2760 | 5740 | 5110 | 4825 | 1406.1 |
| 0.5 | 7560 | 4480 | 8490 | 9260 | 7447.5 | 2096.9 |
| 1 | 7600 | 7320 | Missing | 11400 | 8773.333 | 2279.1 |
| 1.5 | 7150 | 6170 | 10500 | 14900 | 9680 | 3943.0 |
| 2 | 8020 | 11000 | 12500 | 14800 | 11580 | 2841.6 |
| 3 | 6580 | 12900 | 9740 | 14100 | 10830 | 3377.8 |
| 4 | 4610 | 12400 | 6820 | 8660 | 8122.5 | 3297.5 |

TABLE 22
Gabapentin, duodenal dose 20 mg/rat

| Time | rat1 | rat2 | rat3 | rat4 | Average | |
|---|---|---|---|---|---|---|
| (h) | (ng/mL) | (ng/mL) | (ng/mL) | (ng/mL) | (ng/mL) | Std Dev. |
| 0 | 0 | 0 | 0 | 0 | 0 | 0 |
| 0.25 | 5560 | 6720 | 7910 | 8050 | 7060 | 1164.5 |
| 0.5 | 7360 | 9850 | 13100 | 11800 | 10527.5 | 2498.6 |
| 1 | 7970 | 13500 | 13700 | 15800 | 12742.5 | 3347.4 |
| 1.5 | 10300 | 13400 | 13500 | 16200 | 13350 | 2411.8 |
| 2 | 9530 | 12500 | 14100 | 17600 | 13432.5 | 3362.2 |
| 3 | 6530 | 9070 | 10200 | 16900 | 10675 | 4424.7 |
| 4 | 4370 | 5900 | 6050 | 13900 | 7555 | 4297.6 |

FIG. 19A shows the gabapentin plasma concentration, in ng/mL, in the animals treated with neat gabapentin, administered intravenously (triangles) and to the duodenum at dosages of 5 mg (circles), 10 mg (squares) and 20 mg (diamonds). An increasing blood concentration with increasing dose was observed for the animals receiving drug via intubation into the duodenum. Naturally, the lower plasma drug concentration for the animals treated intravenously (triangles) is due to the lower drug dose.

FIG. 19B shows the results for the animals receiving gabapentin-lauryl sulfate complex intravenously (triangles) and directly to the duodenum at dosages of 5 mg (circles), 10 mg (squares), and 20 mg (diamonds). While the absolute blood concentrations of the animals receiving gabapentin-lauryl sulfate complex are lower than the animals treated with gabapentin, the data shows that absorption of gabapentin from the complex is enhanced relative to absorption of the neat drug, due perhaps in part to the L-amino acid transport system not being saturated and/or the increased transport via other mechanisms provided by the complex. This is evident from a comparison of the blood concentration between the 5 mg and 10 mg dose and between the 10 mg and 20 mg dose in FIGS. 6A and 6B, where the increase in blood concentration with increased dose is greater for gabapentin administered in the form of the complex.

FIG. 19C shows the percent bioavailability of gabapentin administered as the neat drug (inverted triangles) or as gabapentin lauryl sulfate complex (circles) to the duodenum of rats. Percent bioavailability is determined relative to gabapentin administered intravenously. At a dosage of 20 mg, gabapentin-lauryl sulfate complex exhibited a higher bioavailability than did the neat drug. The increased bioavailability at the higher doses is likely due to the enhanced absorption offered by the complex, where uptake in the G.I. tract is not limited to uptake by the L-amino acid transport system for the complex, but is also occurring by transcellular and paracellular mechanisms.

Table 23 shows the pharmacokinetic analysis from the study, where the area under curve from 0 to 4 hours was determined, and normalized to a 1 mg does of gabapentin/kg rat. The data relating to the hour 4 point for gabapentin (iv) assumes a log-linear decline from the data measured for the first three hours. Percent bioavailability is relative to the bioavailability of intravenously administered gabapentin.

TABLE 23

| | | AUC | Bio- |
|---|---|---|---|
| | Dose | (0˜4 h, ng · h/ | availability |
| Drug Form | (mg/kg. ± s.d.) | ml-mg ± s.d.)* | (%) |
| gabapentin (iv) | 1 | 2727.1 ± 259.1 | 100.0 |
| gabapentin (duodenal) | 14.8 ± 0.1 | 1705.2 ± 257.2 | 62.5 ± 9.4 |
| gabapentin (duodenal) | 30.6 ± 1.7 | 1205.7 ± 276.3 | 44.2 ± 10.1 |
| gabapentin (duodenal) | 59.8 ± 1.7 | 726.1 ± 223.9 | 26.2 ± 8.2 |
| gabapentin lauryl sulfate | 14.0 ± 0.1 | 1604.3 ± 479.1 | 58.8 ± 17.6 |
| (duodenal) | | | |
| gabapentin lauryl sulfate | 29.1 ± 1.1 | 1182.2 ± 267.9 | 43.3 ± 9.8 |
| (duodenal) | | | |
| gabapentin lauryl sulfate | 58.1 ± 2.3 | 1033.9 ± 88.9 | 37.9 ± 3.3 |
| (duodenal) | | | |

Normalized to dose of 1 mg gabapentin/kg.

The AUC and bioavailability data show that as the dose increases, colonic absorption of gabapentin is improved when the drug is provided in the form of a gabapentin-transport moiety complex.

### Example 11

Preparation of Pregabalin-Transport Moiety Complex

1. A solution of 0.5 mL 36.5% hydrochloric acid (5 mmol HCl) in 25 mL deionized water is prepared.
2. 5 mmol pregabalin (0.80 g) is added to the solution in step 1. The mixture is stirred for 10 min at room temperature. Pregabalin hydrochloride is formed.
3. 5 mmol sodium lauryl sulfate (1.4 g) is added to the aqueous solution in step 2.

The mixture is stirred for 20 min at room temperature.
4. 50 mL dichloromethane is added to the solution in step 3. The mixture is stirred for 2 hours at room temperature.
5. The mixture of step 4 is transferred to a separatory funnel and allowed to settle for 3 hours. Two phases are formed, a lower phase of dichloromethane and an upper phase of water.
6. The upper and lower phases in step 5 are separated. The lower dichloromethane phase is recovered and the dichloromethane is evaporated to dryness at room temperature, followed by drying in a vacuum oven for 4 hours at 40° C. A complex of pregabalin-lauryl sulfate (2.1 g) is obtained.

FIG. 16D shows a synthetic reaction scheme for preparation of a pregabalin-alkyl sulfate complex.

### Example 12

In Vivo Colonic Absorption Using Flushed Ligated Colonic Model in Rats

An animal model commonly known as the “intracolonic ligated model” is employed. Fasted, 0.3-0.5 kg Sprague-Dawley male rats are anesthetized and a segment of proximal colon is isolated. The colon is flushed of fecal materials. The segment is ligated at both ends while a catheter is placed in the lumen and exteriorized above the skin for delivery of test formulation. The colonic contents are flushed out and the colon is returned to the abdomen of the animal. Depending on the experimental set up, the test formulation is added after the segment is filled with 1 mL/kg of 20 mM sodium phosphate buffer, pH 7.4, to more accurately simulate the actual colon environment in a clinical situation.

Rats (n=3) are allowed to equilibrate for approximately 1 hour after surgical preparation and prior to exposure to each test formulation. Pregabalin-lauryl sulfate complex or pregabalin are administered as an intracolonic bolus and delivered at 10 mg pregabalin/rat. Blood samples obtained from the jugular catheter are taken at 0, 15, 30, 60, 90, 120, 180 and 240 minutes for analysis of pregabalin concentration. At the end of the 4 hour test period, the rats are euthanized with an overdose of pentobarbital. Colonic segments from each rat are excised and opened longitudinally along the anti-mesenteric border. Each segment is observed macroscopically for irritation and any abnormality noted. The excised colons are placed on graph paper and measured to approximate colonic surface area.

A control group of rats (n=3) is treated with pregabalin intravenously, at a dose of 1 mg/rat. Blood samples are withdrawn at the same times indicated above.

### Example 13

In Vivo Absorption of Pregabalin

Twenty-eight rats are randomized into seven test groups (n=4). Pregabalin or pregabalin-lauryl sulfate complex, prepared as described in Example 1B, in water is intubated via catheter into the beginning of the duodenum of rats at dosages of 5 mg/rat, 10 mg/rat, and 20 mg/rat. The remaining test group is given 1 mg/kg pregabalin intravenously.

Blood samples are taken from each animal over a four hour period and analyzed for pregabalin content. The dose, AUC, and bioavailability are determined using similar calculations as used for gabapentin in Example 10.

Iron

The term “iron” comprises iron (Fe) in any of its oxidative states and in combination with any salt. “Ferrous” refers to iron with a +2 charge (also denoted in the art as Fe2+, Fe++, iron (II)). “Ferric” refers to iron with a +3 charge (also denoted in the art as Fe3+, Fe+++, iron (III)). Exemplary ferrous salts and ferric salts include, but are not limited to ferrous and ferric sulfate, fumarate, succinate, gluconate, etc.

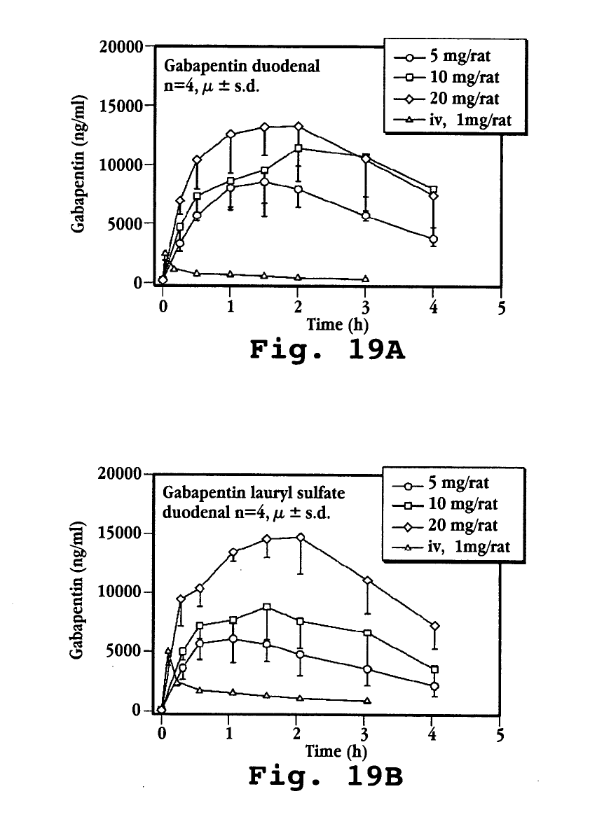

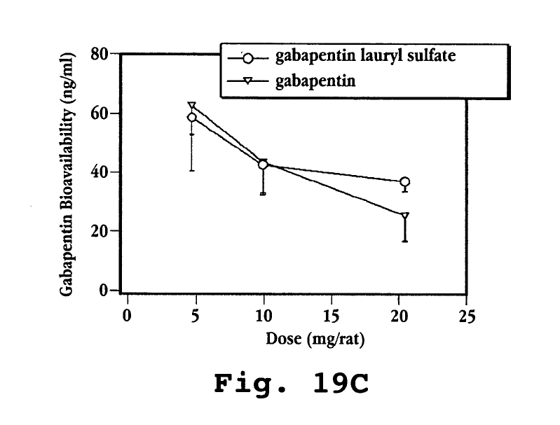

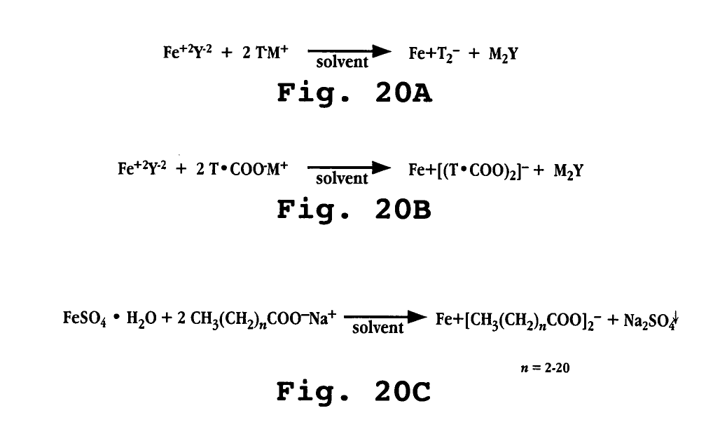

FIG. 20 shows a synthetic reaction scheme for preparation of an iron-fatty acid complex.

### Example 14

Preparation of Iron-Fatty Acid Complex

The following steps are carried out to form a ferrous-fatty acid complex. The reaction is illustrated in FIGS. 20A-20C.
1. 9.15 grams of FeSO4.7H2O were dissolved into 300 mL methanol in a beaker.
2. 14.64 grams of lauric acid sodium (sodium laurate) were dissolved into 300 mL methanol in a second beaker.
3. The solution of step 1 was added dropwise to the solution of step 2. The mixture was stirred for 1-5 h at room temperature to produce a precipitate of Na2SO4. The solution was stirred overnight.
4. The precipitate from step 3 was removed via vacuum filtration using with #42 Whatman filter paper; the filtrate was captured in a funnel. The precipitate washed three times with methanol; the filtrate was captured into the funnel.
5. The filtrate solution of step 4 was placed in a crystallizing dish and placed in a hood to evaporate the solvent. A beige precipitate formed. The precipitate was placed on a vacuum filter and the remaining solvent was removed via vacuum filtration. The filter cake was placed in a crystallizing dish and placed in a vacuum oven overnight to dry.

The melting point of the precipitate was determined to be between 38-38° C.

### Example 15

In Vivo Colonic Absorption Using Flushed Ligated Colonic Model in Rats

The lower G.I. absorption and bioavailability of iron-transport moiety complexes is evaluated using an animal model commonly known as the “intracolonic ligated model”. Surgical preparation of a fasted anesthetized 0.3-0.5 kg Sprague-Dawley male rats proceeds as follows. A segment of proximal colon is isolated and the colon is flushed of fecal materials. The segment is ligated at both ends while a catheter is placed in the lumen and exteriorized above the skin for delivery of test formulations. The colonic contents are flushed out and the colon is returned to the abdomen of the animal. Depending on the experimental set up, the test formulation is added after the segment is filled with 1 mL/kg of 20 mM sodium phosphate buffer, pH 7.4, to more accurately simulate the actual colon environment in a clinical situation.

Rats are allowed to equilibrate for approximately 1 hour after surgical preparation and prior to exposure to each iron-transport moiety complex. The test compounds are administered as an intracolonic bolus and delivered at 10 mg iron (as Fe+2/rat). Blood samples are obtained from the jugular catheter were at 0, 15, 30, 60, 90, 120, 180 and 240 minutes and are analyzed for blood iron concentration. At the end of the 4 hour test period, the rats are euthanized with an overdose of pentobarbital. Colonic segments from each rat are excised and opened longitudinally along the anti-mesenteric border. Each segment is observed macroscopically for irritation and any abnormality noted. The excised colons are placed on graph paper and measured to approximate colonic surface area.

The above procedure is used to evaluate the absorption of ferrous sulfate salt, and of ferrous-laurate complex, ferrous-caprate complex, ferrous-oleate complex, and ferrous-palmitate complex.

### Example 16

Twenty-eight rats are randomized into seven test groups (n=4). Ferrous sulfate or ferrous-laurate complex, prepared as described in Example 11, is intubated via catheter into the beginning of the duodenum of rats at dosages of 5 mg/rat, 10 mg/rat, and 20 mg/rat. The remaining test group is given 1 mg/kg ferrous-sulfate intravenously.

Dipeptidyl Peptidase IV Inhibitors

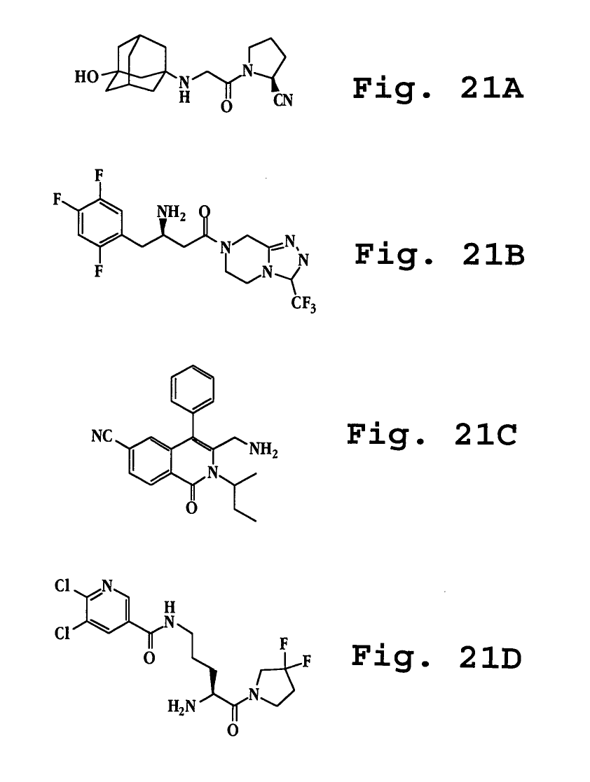

DPP IV inhibitors are compounds that inhibit the enzymatic activity of DPP-IV, however the compounds may also have inhibitory activity on other DPP enzymes. A vast number of DPP-IV inhibitors have been identified, and four exemplary compounds are shown in FIGS. 21A-21D.

FIG. 21A shows the structure of the DPP IV inhibitor 1-[[(3-hydroxy-1-adamantyl)amino]acetyl]-2-cyano-(S)-pyrrolidine, a compound identified as LAF-237 (Villhauer, E. B. et al., Journal of Medicinal Chemistry, 46, 2774-2789 (2003)). FIG. 21B shows the structure of an aminoacyl triazolopyrazine DPP IV inhibitor described in detail in WO2004032836, incorporated by reference herein. FIG. 21C shows the structure of another exemplary DPP IV inhibitor, described in WO2004/024184. FIG. 21D shows the structure of a DPP IV inhibitor, 1-[N-(5,6-dichloronicotinoyl)-L-ornithinyl]-3,3-difluoropyrrolidine hydrochloride, described in WO03/000250, which is incorporated by reference herein. This compound is also referred to herein as “the difluoropyrrolidine compound.”

Complexes with DPPIV inhibitors are prepared as follows.

### Example 17

Preparation of DPP IV-Inhibitor-Fatty Acid Complexes

Preparation of Complex Using the Difluoropyrrolidine DPP IV Inhibitor

An oleic acid-acetone solution, 16.0 g oleic acid dissolved in 100 mL acetone, is prepared.

22.0 grams of the DPP IV inhibitor as the free base identified as the difluoropyrrolidine compound (FIG. 21D) are dissolved in 200 mL acetone.

The oleic acid-acetone solution is added dropwise to the solution containing the DPP IV inhibitor, with stirring. Stirring is continued overnight at ambient temperature (20-25° C.). A difluoropyrrolidine compound-oleate complex precipitates.

The mixture of solvent and precipitated difluoropyrrolidine compound-oleate complex is filtered through a Buchner funnel. The filter cake is rinsed with 4×200 mL acetone and then is dried under vacuum suction for an hour. The filter cake is scraped off the filter paper and weighed.

Preparation of Complex Using a Cyanopyrrolidine DPP IV Inhibitor

1. An oleic acid-acetone solution, 16.0 g oleic acid dissolved in 100 mL acetone, is prepared.
2. 16.9 grams of the DPP IV inhibitor as the free base identified as the cyanopyrrolidine compound (FIG. 21A) are dissolved in 200 mL acetone.
3. The oleic acid-acetone solution is added dropwise to the solution containing the DPP IV inhibitor, with stirring. Stirring is continued overnight at ambient temperature (20-25° C.). A cyanopyrrolidine compound-oleate complex is formed.
4. The cyanopyrrolidine compound-oleate complex is recovered from the solution using a suitable technique, such as filtration or extraction, depending on the form of the complex.
   Preparation of Complex Using a Homophenylalanine DPP IV Inhibitor

An oleic acid-acetone solution, 16.0 g oleic acid dissolved in 100 mL acetone, is prepared.

22.7 grams of the DPP IV inhibitor as the free base identified as the homophenylalanine compound (FIG. 21B) are dissolved in 200 mL acetone.

The oleic acid-acetone solution is added dropwise to the solution containing the DPP IV inhibitor, with stirring. Stirring is continued overnight at ambient temperature (20-25° C.). A homophenylalanine compound-oleate complex is formed.

The homophenylalanine compound-oleate complex is recovered from the solution using a suitable technique, such as filtration or extraction, depending on the form of the complex.

What is claimed is:

1. A substance comprising:
a complex that comprises a drug moiety, and a transport moiety.

2. The substance of claim 1, wherein the transport moiety comprises an acidic, a basic, or a zwitterionic structural element; or an acidic, a basic, or a zwitterionic residual structural element paired with an ion to form a salt.

3. The substance of claim 2, wherein the transport moiety comprises fatty acids or its salts, benzenesulfonic acid or its salts, benzoic acid or its salts, fumaric acid or its salts, or salicylic acid or its salts.

4. The substance of claim 3, wherein the fatty acids or its salts comprises a C6-C18 fatty acid or its salt.

5. The substance of claim 4, wherein the C6-C18 fatty acid or its salt comprises a C12 fatty acid or its salt.

6. The substance of claim 2, wherein the transport moiety comprises an alkyl sulfate or its salt.

7. The substance of claim 6, wherein the alkyl sulfate or its salt comprises a C6-C18 sodium alkyl sulfate or its salt.

8. The substance of claim 7, wherein the C6-C18 sodium alkyl sulfate or its salt comprises sodium lauryl sulfate.

9. The substance of claim 2, wherein the transport moiety comprises a pharmaceutically acceptable primary, secondary, or tertiary amine, or salts thereof.

10. The substance of claim 1, wherein the drug moiety comprises an acidic, a basic, or a zwitterionic structural element; or an acidic, a basic, or a zwitterionic residual structural element paired with an ion to form a salt.

11. A composition comprising the substance of claim 1 and an inactive ingredient.

12. A dosage form comprising the composition of claim 11.

13. A method of treating a disease or condition comprising:
administering the substance of claim 1 to a patient need thereof.

14. The method of claim 13, wherein the substance is administered via an oral, intravenous, subcutaneous, intramuscular, transdermal, intraarterial, intraarticular, or intradermal route.

15. A method of making a composition comprising:
providing a drug moiety in an ionic form;
providing a transport moiety in an ionic form;
combining the drug moiety and the transport moiety, in the presence of a solvent having a dielectric constant less than that of water, to form a complex; and
separating the complex from the solvent.

16. The method of claim 15, wherein the transport moiety comprises an acidic, a basic, or a zwitterionic structural element; or an acidic, a basic, or a zwitterionic residual structural element paired with an ion to form a salt.

17. The method of claim 15, wherein the drug moiety comprises an acidic, a basic, or a zwitterionic structural element; or an acidic, a basic, or a zwitterionic residual structural element paired with an ion to form a salt.

18. The method of claim 17, wherein the drug moiety comprises an acidic structural element or an acidic residual structural element; and the drug moiety is processed to obtain the acid form of the drug moiety.

19. The method of claim 17, wherein the drug moiety comprises a basic structural element or a basic residual structural element; and the drug moiety is processed to obtain the basic form of the drug moiety.

20. The method of claim 15, wherein the drug moiety comprises a zwitterionic structural element or zwitterionic residual structural element; and a non-bonding structural element or residual structural element of the zwitterionic structural element or zwitterionic residual structural element is blocked before reacting the drug moiety and the transport moiety.

21. A method of treatment comprising:
providing a drug moiety in an ionic form;
providing a transport moiety in an ionic form;
combining the drug moiety and the transport moiety, in the presence of a solvent having a dielectric constant less than that of water, to form a complex;
separating the complex from the solvent; and
administering the separated complex to a patient in need thereof.

22. The method of claim 21, wherein the transport moiety comprises an acidic, a basic, or a zwitterionic structural element; or an acidic, a basic, or a zwitterionic residual structural element paired with an ion to form a salt.

23. The method of claim 21, wherein the drug moiety comprises an acidic, a basic, or a zwitterionic structural element; or an acidic, a basic, or a zwitterionic residual structural element paired with an ion to form a salt.

24. The method of claim 23, wherein the drug moiety comprises an acidic structural element or an acidic residual structural element; and the drug moiety is processed to obtain the acid form of the drug moiety.

25. The method of claim 23, wherein the drug moiety comprises a basic structural element or a basic residual structural element; and the drug moiety is processed to obtain the basic form of the drug moiety.

26. The method of claim 23, wherein the drug moiety comprises a zwitterionic structural element or zwitterionic residual structural element; and a non-bonding structural element or residual structural element of the zwitterionic structural element or zwitterionic residual structural element is blocked before reacting the drug moiety and the transport moiety.

27. The method of claim 21, wherein the complex is administered via an oral, intravenous, subcutaneous, intramuscular, transdermal, intraarterial, intraarticular, or intradermal route.

28. A method of improving absorption of a drug moiety comprising:
providing a complex of the drug moiety and a transport moiety; and
administering the complex to a patient in need thereof.

29. The method of claim 28, wherein the complex is administered orally, and the improved absorption comprises improved oral absorption.

30. The method of claim 29, wherein the improved oral absorption comprises improved lower gastrointestinal tract absorption.

31. The method of claim 29, wherein the improved oral absorption comprises improved upper gastrointestinal tract absorption.

32. The method of claim 28, wherein the complex is administered transdermally, and the improved absorption is improved transdermal absorption.

33. The method of claim 28, wherein the complex is administered subcutaneously, and the improved absorption is improved subcutaneous absorption.
